(19) United States
(12) Patent Application Publication
(10) Pub. No.: US 2020/0209247 A1
(43) Pub. Date: Jul. 2, 2020

(54) METHODS FOR IDENTIFYING MYELOMA TUMOR-INITIATING CELLS AND TARGETED THERAPY

(71) Applicant: UNIVERSITY OF IOWA RESEARCH FOUNDATION, Iowa City, IA (US)

(72) Inventors: Ivana Frech, Iowa City, IA (US); Fenghuang Zhan, Iowa City, IA (US); Guido Tricot, Iowa City, IA (US)

(73) Assignee: UNIVERSITY OF IOWA RESEARCH FOUNDATION, Iowa City, IA (US)

(21) Appl. No.: 16/644,106

(22) PCT Filed: Sep. 17, 2018
(86) PCT No.: PCT/US2018/051363

§ 371 Date: Mar. 3, 2020

Related U.S. Application Data
(60) Provisional application No. 62/559,272, filed on Sep. 15, 2017.
(60) Provisional application No. 62/593,717, filed on Dec. 1, 2017.

(51) Int. Cl. G01N 33/574; C07K 14/705; C07K 16/28; A61P 35/00

(52) U.S. Cl. CPC G01N 33/57492; C07K 14/70596; A61K 38/00; A61P 35/00; G01N 2333/70596; C07K 16/2896

(57) ABSTRACT

In certain embodiments, the present invention provides a method of treating cancer in a patient comprising administering an effective amount of a therapeutic agent to the patient, wherein the cancer was determined to contain cells comprising cell marker CD24⁺. In certain embodiments, the cancer cells also comprise cell markers CD38⁺ and/or CD45⁻.

### PRIORITY APPLICATION

This application claims priority to U.S. Provisional Application No. 62/559,272 that was filed on Sep. 15, 2017 and U.S. Provisional Application No. 62/593,717 that was filed on Dec. 1, 2017. The entire contents of the application referenced above is hereby incorporated by reference herein.

### BACKGROUND

Multiple Myeloma (MM) is a bone marrow (BM) malignancy of the B-cell lineage characterized by monoclonal plasma cells (PCs) in BM. Despite the recent advances in therapy, MM remains incurable and accounts for 19% of deaths from hematopoietic malignancies. Most patients initially respond to therapy, but nearly all relapse and become refractory to treatment. Treatment failure is due to persistence of a minor population of cancer stem or tumor-initiating cells that are non-cycling or low-cycling, and drug-resistant tumor cells. To-date, it is not possible to identify this population.

The functional features of MM-initiating tumor cells, such as drug resistance and self-renewal, play an important role in dictating clinical outcomes. The understanding of MM-initiating tumor biology may lead to the development of novel prognostic and therapeutic targets that can be tested in both preclinical and clinical studies. Finally, clinical trials and correlative studies should provide evidence regarding the inhibition of MM-initiating tumors, leading to improvements in long-term clinical outcomes.

Therefore, there is an on-going need for the identification of MM-initiating tumor markers.

### SUMMARY

In certain embodiments, the present invention provides a method of treating cancer in a patient comprising administering an effective amount of a therapeutic agent to the patient, wherein the cancer was determined to contain cells comprising cell marker CD24⁺.

In certain embodiments, the present invention provides a method of treating a cancer cell comprising cell marker CD24⁺, the method comprising administering to the cell a therapeutic agent.

In certain embodiments, the present invention provides a method for predicting whether a cancer will respond to a therapeutic agent that targets a protein in the PBK pathway, the method comprising: (i) obtaining a test sample from a subject, wherein said test sample is from the cancer; (ii) assaying the test sample for the presence of cell marker CD24⁺; and (iii) using the presence of the cell marker CD24⁺ to indicate that the cancer will respond to the therapeutic agent that targets.

### BRIEF DESCRIPTION OF THE FIGURES

FIGS. 1A-1C. The long-term remission (>10 yrs.) MM samples contain abnormal genetic signatures. (FIG. 1A) A 2-dimensional unsupervised hierarchical cluster analysis of 52 genes (rows) in CD138-enriched plasma cells from normal plasma cells (NPC; n=22), patients with MGUS (n=44), MM samples from long-remission survivors in TT1 cohort (n=20), newly diagnosed MM from TT2 (n=351) and TT3 (n=206). (FIG. 1B) The Bar-view shows the spike expression of chromosomal translocation-activated genes from the corresponding samples described in A. (FIG. 1C) Spike genes' expression in relapsed MM samples. Bar-view shows the translocation-activated genes' expression in 51 patients with paired samples collected at diagnosis and at relapse from the same patient.

FIGS. 2A-2F. Identification of surface markers differentially expressed in side-population MM cells. (FIG. 2A) An unsupervised hierarchical clustering analysis presented genes differentially expressed between side-population+/light chain restricted (SP+/LC) with CD138⁺ MM cells. Red color and green color represent gene expression above or lower than the mean value. Red arrow is labelled the expression of CD24. (FIG. 2B) Bar-view showed the CD24 expression in seven paired samples isolated by SP⁺/LC or CD138⁺ antibodies from Affymetrix microarrays. (FIG. 2C) CD24 mRNA expression from four MM patients with paired SP⁺/LC and CD138⁺ MM cells were detected by q-PCR. (FIG. 2D) Flow-cytometry analyzed CD24⁺ population in MM cell lines. The Figure showed a representative ARP1 MM cell line. (FIG. 2E) The expression of iPS/ES genes was compared between CD24⁺ and CD24− MM cell lines ARP1 by qRT-PCR. (FIG. 2F) The expression of iPS/ES genes was compared between CD24⁺/LC MM cells with the CD138⁺ MM cells by qRT-PCR.

FIGS. 3A-3G. CD24+ MM cells are enriched in MM samples after treatment. (FIG. 3A) Flow cytometry showed a representative sample analyzed by CD138, CD38 and CD24 antibodies. (FIG. 3B) The correlation between CD138⁺/CD38⁺ percentage with CD138⁺/CD24⁺ percentage was analyzed in 48 MM cases. (FIGS. 3C˜F) The percentages of CD24⁺ MM cells were compared in MM patients with or without treatment FIG. 3C), in partial remission (PR) or complete remission (CR) (FIG. 3D), in different clinical stages (FIG. 3E), and with or without bone lytic lesions (FIG. 3F). (FIG. 3G) q-RT PCR detected the expression of CD24 and iPS/EG genes in CD24⁺ or CD24⁻ MM cells isolated from primary MM samples.

FIGS. 4A-4H. CD24⁺ MM cells showed strong clonogenicity and tumorigenicity. CD24⁺ and CD24⁻ cells from ARP1 cell line (FIGS. 4A & B) and OCI-MY5 cell line (FIGS. 4C & D) were serially plated in methylcellulose with triplicate up to three passages, respectively. The colony quantification was shown in the right panels for each passage. (FIGS. 4E˜H) Representative IVIS showed the tumor growth from the 1st, 2^{nd}, and 3^{rd} transplantations of ARP1 MM cells. Right flanks were injected with CD24⁺ ARP1 cells and the left flanks were injected with CD24⁻ ARP1 cell.

FIGS. 5A-5F. CD24⁺ MM cells was more resistant to chemotherapeutic drugs than those CD24⁻ counterparts. CD24⁺ and CD24⁻ ARP1 cells (FIG. 5A) and OCI-MY5 (FIG. 5C) in the 2^{nd} passage were plated for colony formation and treated with indicated drugs and different doses. The colony quantification was shown in the right panels for each drug respectively (FIG. 5B & FIG. 5D). (FIG. 5E) Flow cytometry indicated of CD24⁺ population in ARP1, RPMI-8226 and RPMI-8226-R5. (FIG. 5F) CD24 expression analyzed by RT-PCR in survival cells compared to the untreated MM cells

FIGS. 6A-6E. STAT3 was the major pathway activated in CD24⁺ MM cells. (FIG. 6A) A heatmap showed the top significantly expressed genes between CD24⁺ and CD24⁻ MM cell lines. (FIG. 6B) GSEA showed the top enriched signaling pathways in CD24⁺ MM cell lines. We noticed that the STAT3 pathway was the most activated signaling in CD24⁺ MM cells. (FIG. 6C) The nuclear protein from CD24⁺ and CD24⁻ populations of ARP1 cells were extracted. H2B, GAPDH and β-Actin were detected to confirm that nuclear protein was extracted successfully by Western blot. (FIG. 6D) The nuclear extracts of CD24⁺ and CD24⁻ cells were used for the analysis of cancer stem cell TF activity. Bar-view showed the activity of 23 transcription factors between CD24⁺ and CD24⁻ populations. (FIG. 6E) The total STAT3 and activated STAT3 (p-STAT3) proteins were detected in CD24⁺ and CD24⁻ ARP1 MM cells by western blots.

FIGS. 7A-7H. STAT3 mediates the tumor-initiating cell features in CD24⁺ MM cells. (FIG. 7A) The 2^{nd} passage ARP1 MM cells transfected with inducible STAT3 shRNA were plated in soft-agar plates. Doxycyline was added to knockdown of STAT3 after plating. The quantification of colony number was evaluated in 3 week (FIG. 7B). (FIG. 7C) About 0.5×10⁶ ARP cells expressing STAT3 shRNA were injected into NOD-Rag1^{null} mice subcutaneously. Half of the mice (n=3) received added doxycline in the drinking water for 31 days. (FIG. 7D) Tumors from mice described in B were harvested and photographed. (FIG. 7E) Quantifications of tumors volume from dissected tumors in C. (FIG. 7F) Quantifications of tumors weight from dissected tumors in C. (FIG. 7G) Western blots showed the expression of STAT3 from dissected tumors described in C. (FIG. 7H) The mRNA expression of CD24, STAT3, NANOG, SOX 2 and OCT4 were detected in dissected tumors described in C.

FIG. 8: 10 CD24⁺ cells initiate tumor in vivo. The CD24⁺ (right flank) and CD24⁻ (left flank) MM cells were injected into NOD-SCID mice.

### DETAILED DESCRIPTION

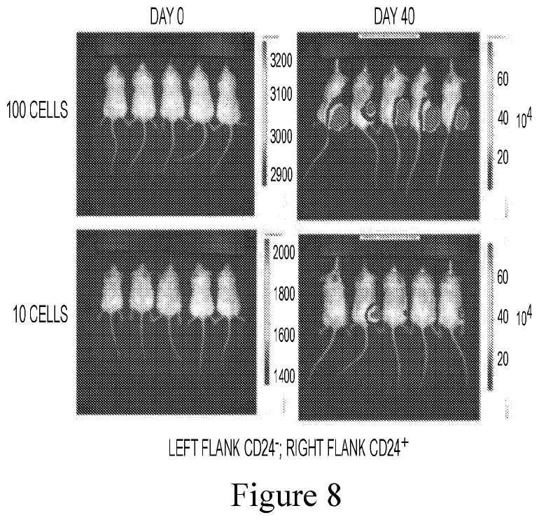

From a systemic analysis of primary MM samples and MM cell lines using gene expression profiles, it has been discovered that CD24⁺ MM cells have a potential MM-initiating tumor marker. The human cell surface antigen CD24 is a sialoglycoprotein localized in membrane lipid raft domains and is a heat-stable antigen. CD24 is used as a marker to differentiate hematopoietic cells and neuronal cells and B lymphocytes. It is also expressed on normal monocytes, granulocytes, red blood cells, platelets and activated T lymphocytes. Recently, many studies have indicated that CD24 is expressed and has been recognized as a sialoglycoprotein marker in multiple cancers. The human CD24 peptide contains 32 residues and activates multiple signaling pathways, such as MAPK signaling, NF-kB signaling, Notch and Hedgehog signaling, which are active in MM. The present data demonstrate that CD24⁺ MM cells showed increased clonogenic potential and drug resistance in vitro and tumorigenesis in vivo after injecting only ten cells from MM cell lines (FIG. 8).

CD38 is highly expressed in MM cells and is widely recognized as a MM cell marker. It recently was successfully used in the treatment of MM.

CD45 is present in a subset of primary MM cells. CD45-negative primary MM cells are quiescent and drug resistant. The present data indicate that CD24⁺ primary MM cells are CD45⁻. Therefore, MM cells having the markers CD38⁺/CD45⁻/CD24⁺ are MM-initiating tumor cells, and the markers CD38⁺/CD45⁻/CD24⁺ can be used in a flow cytometry panel.

The present discovery is innovative and the first one that addressed the hypothesis that CD24⁺ is a key marker for MM-initiating tumors, allowing for a significant improvement in the clinical outcome of MM and on other tumors that initiate with CD24⁺ cells.

Current MM diagnostic tools include CD38⁺/CD45⁻/CD138⁺ Cytoplasmic kappa and lambda. This flow cytometry panel allows to quantify the percentage of bulk myeloma cells does not provide information on MM-initiating tumor cells. Other diagnostic tools include blood tests for tumor burden, liver, kidney, etc. functions, and X-rays, MRI, CT, etc. to assess bone damage. These tools, however, also do not provide information regarding MM-initiating tumor cells.

Advantages of CD38⁺/CD45⁻/CD24⁺ Diagnostic Tool

Currently, no phenotypic markers are known for identifying tumor-initiating cells in multiple myeloma, and there is an on-going need to identify these tumor-initiating cells in order that proper therapies can be administered. CD24⁺ positive myeloma cells represent a population related to drug resistance and poor prognosis. Therefore, quantification of this population from bone marrow aspirates or peripheral blood samples could be value for disease progression.

Methods of Treating Myeloma

CD24⁺ is a cell surface protein and can be easily targeted by humanized anti-CD24 antibody, siRNA, oligo nucleotides, small chemical compounds, etc. Targeting CD24⁺ myeloma cells may eliminate tumor-initiating cells resulting in cure myeloma disease.

The inventors have identified that STAT3 is a downstream target of CD24. The STAT3 inhibitor, which is used in clinical trial, inhibits CD24⁺ MM-initiating cell growth.

Further, P-selectin is the ligand of CD24. P-selectin antibodies are used in clinical for treating anemia. The P-selectin antibody can be used to kill CD24⁺ myeloma-initiating cells.

CD24⁺ plasma cells are persistent in pre-myeloma diseases, such as monoclonal gammopathy of undetermined significance (MGUS) and smoldering multiple myeloma (SMM). Monitoring CD24⁺ plasma cells in MGUS and SMM can predict disease progress from a benign disease to a malignancy. Accordingly, as a preventive, targeting CD24⁺ can prevent the disease transitioning from MGUS or SMM to symptomatic myeloma disease. The term “detection” includes any means of detecting, including direct and indirect detection.

The term “diagnosis” is used herein to refer to the identification or classification of a molecular or pathological state, disease or condition. For example, “diagnosis” may refer to identification of a particular type of cancer, e.g., a lung cancer. “Diagnosis” may also refer to the classification of a particular type of cancer, e.g., by histology (e.g., a non-small cell lung carcinoma), by molecular features (e.g., a lung cancer characterized by nucleotide and/or amino acid variation(s) in a particular gene or protein), or both.

The term “prognosis” is used herein to refer to the prediction of the likelihood of cancer-attributable death or progression, including, for example, recurrence, metastatic spread, and drug resistance, of a neoplastic disease, such as cancer.

The term “prediction” or (and variations such as predicting) is used herein to refer to the likelihood that a patient will respond either favorably or unfavorably to a drug or set of drugs.

In one embodiment, the prediction relates to the extent of those responses. In another embodiment, the prediction relates to whether and/or the probability that a patient will survive following treatment, for example treatment with a particular therapeutic agent and/or surgical removal of the primary tumor, and/or chemotherapy for a certain period of time without cancer recurrence. The predictive methods of the invention can be used clinically to make treatment decisions by choosing the most appropriate treatment modalities for any particular patient. The predictive methods of the present invention are valuable tools in predicting if a patient is likely to respond favorably to a treatment regimen, such as a given therapeutic regimen, including for example, administration of a given therapeutic agent or combination, surgical intervention, chemotherapy, etc., or whether long-term survival of the patient, following a therapeutic regimen is likely.

The terms “cell proliferative disorder” and “proliferative disorder” refer to disorders that are associated with a measurable degree of abnormal cell proliferation. In one embodiment, the cell proliferative disorder is cancer.

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder” and “tumor” are not mutually exclusive as referred to herein.

The terms “cancer” and “cancerous” refer to or describe the physiological condition in mammals that is typically characterized by unregulated cell growth and proliferation. Examples of cancer include, but are not limited to, carcinoma, lymphoma (e.g., Hodgkin's and non-Hodgkin's lymphoma), blastoma, sarcoma, and leukemia. More particular examples of cancers include squamous cell cancer, small-cell lung cancer, non-small cell lung cancer, adenocarcinoma of the lung, squamous carcinoma of the lung, cancer of the peritoneum, hepatocellular cancer, renal cell carcinoma, gastrointestinal cancer, gastric cancer, esophageal cancer, pancreatic cancer, glioma, cervical cancer, ovarian cancer, liver cancer, bladder cancer, hepatoma, breast cancer (e.g., endocrine resistant breast cancer), colon cancer, rectal cancer, lung cancer, endometrial or uterine carcinoma, salivary gland carcinoma, kidney cancer, liver cancer, prostate cancer, vulval cancer, thyroid cancer, hepatic carcinoma, melanoma, leukemia and other lymphoproliferative disorders, and various types of head and neck cancer. As used herein, “treatment” (and variations such as “treat” or “treating”) refers to clinical intervention in an attempt to alter the natural course of the individual or cell being treated, and can be performed either for prophylaxis or during the course of clinical pathology. Desirable effects of treatment include preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

An “individual,” “subject” or “patient” is a vertebrate. In certain embodiments, the vertebrate is a mammal. Mammals include, but are not limited to, farm animals (such as cows), sport animals, pets (such as cats, dogs, and horses), primates (including human and non-human primates), and rodents (e.g., mice and rats). In certain embodiments, a mammal is a human and can be either a male or a female human.

An “effective amount” refers to an amount effective, at dosages and for periods of time necessary, to achieve the desired therapeutic or prophylactic result.

A “therapeutically effective amount” of a substance/molecule of the invention may vary according to factors such as the disease state, age, sex, and weight of the individual, and the ability of the substance/molecule, to elicit a desired response in the individual. A therapeutically effective amount encompasses an amount in which any toxic or detrimental effects of the substance/molecule are outweighed by the therapeutically beneficial effects. A “prophylactically effective amount” refers to an amount effective, at dosages and for periods of time necessary, to achieve the desired prophylactic result. Typically, but not necessarily, since a prophylactic dose is used in subjects prior to or at an earlier stage of disease, the prophylactically effective amount would be less than the therapeutically effective amount.

The term “long-term” survival is used herein to refer to survival for at least 1 year, 5 years, 8 years, or 10 years following therapeutic treatment.

The term “increased resistance” to a particular therapeutic agent or treatment option, when used in accordance with the invention, means decreased response to a standard dose of the drug or to a standard treatment protocol.

The term “decreased sensitivity” to a particular therapeutic agent or treatment option, when used in accordance with the invention, means decreased response to a standard dose of the agent or to a standard treatment protocol, where decreased response can be compensated for (at least partially) by increasing the dose of agent, or the intensity of treatment.

“Patient response” can be assessed using any endpoint indicating a benefit to the patient, including, without limitation, (1) inhibition, to some extent, of tumor growth, including slowing down or complete growth arrest; (2) reduction in the number of tumor cells; (3) reduction in tumor size; (4) inhibition (e.g., reduction, slowing down or complete stopping) of tumor cell infiltration into adjacent peripheral organs and/or tissues; (5) inhibition (e.g., reduction, slowing down or complete stopping) of metastasis; (6) enhancement of anti-tumor immune response, which may, but does not have to, result in the regression or rejection of the tumor; (7) relief, to some extent, of one or more symptoms associated with the tumor; (8) increase in the length of survival following treatment; and/or (9) decreased mortality at a given point of time following treatment.

“Antibodies” (Abs) and “immunoglobulins” (Igs) refer to glycoproteins having similar structural characteristics. While antibodies exhibit binding specificity to a specific antigen, immunoglobulins include both antibodies and other antibody-like molecules that generally lack antigen specificity. Polypeptides of the latter kind are, for example, produced at low levels by the lymph system and at increased levels by myelomas.

The terms “antibody” and “immunoglobulin” are used interchangeably in the broadest sense and include monoclonal antibodies (e.g., full length or intact monoclonal antibodies), polyclonal antibodies, monovalent antibodies, multivalent antibodies, multispecific antibodies (e.g., bispecific antibodies so long as they exhibit the desired biological activity) and may also include certain antibody fragments (as described in greater detail herein). An antibody can be chimeric, human, humanized and/or affinity matured.

A biological sample, according to any of the above methods, may be obtained using certain methods known to those skilled in the art. Biological samples may be obtained from vertebrate animals, and in particular, mammals. Tissue biopsy is often used to obtain a representative piece of tumor tissue. Alternatively, tumor cells can be obtained indirectly in the form of tissues or fluids that are known or thought to contain the tumor cells of interest. For instance, samples of lung cancer lesions may be obtained by resection, bronchoscopy, fine needle aspiration, bronchial brushings, or from sputum, pleural fluid or blood. Variations in target nucleic acids (or encoded polypeptides) may be detected from a tumor sample or from other body samples such as urine, sputum or serum. Cancer cells are sloughed off from tumors and appear in such body samples. By screening such body samples, a simple early diagnosis can be achieved for diseases such as cancer. In addition, the progress of therapy can be monitored more easily by testing such body samples for variations in target nucleic acids (or encoded polypeptides). Additionally, methods for enriching a tissue preparation for tumor cells are known in the art. For example, the tissue may be isolated from paraffin or cryostat sections. Cancer cells may also be separated from normal cells by flow cytometry or laser capture microdissection.

Methods of Treating

In certain embodiments, the present invention provides a method of treating cancer in a patient comprising administering an effective amount of a therapeutic agent to the patient, wherein the cancer was determined to contain cells comprising cell marker CD24⁺.

In certain embodiments, the present invention provides a method of treating a cancer cell comprising cell marker CD24⁺, the method comprising administering to the cell a therapeutic agent.

In certain embodiments, the cells further comprise cell marker CD38⁺ and/or is CD45 negative.

In certain embodiments, the cancer is multiple myeloma.

In certain embodiments, the therapeutic agent is a STAT3 inhibitor.

In certain embodiments, the therapeutic agent is a ligand of CD24.

In certain embodiments, the ligand of CD24 is a P-selectin antibody.

In certain embodiments, the present invention provides a method for predicting whether a cancer will respond to a therapeutic agent that targets a protein in the PBK pathway, the method comprising: (i) obtaining a test sample from a subject, wherein said test sample is from the cancer; (ii) assaying the test sample for the presence of cell marker CD24⁺; and (iii) using the presence of the cell marker CD24⁺ to indicate that the cancer will respond to the therapeutic agent.

In certain embodiments, the cells further comprise cell marker CD38⁺ and/or are CD45 negative.

In certain embodiments, the cancer is multiple myeloma.

In certain embodiments, the assay is flow cytometry.

In certain embodiments, the method further comprises administering to a human subject having the cancer an effective amount of a therapeutic agent.

In certain embodiments, the therapeutic agent is a STAT3 inhibitor.

In certain embodiments, the therapeutic agent is a ligand of CD24.

In certain embodiments, the ligand of CD24 is a P-selectin antibody.

The use of the terms “a” and “an” and “the” and similar terms in the context of describing embodiments of invention are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. In addition to the order detailed herein, the methods described herein can be performed in any suitable order unless otherwise indicated herein or otherwise clearly contradicted by context. The use of any and all examples, or exemplary language (e.g., “such as”) provided herein, is intended merely to better illuminate embodiments of invention and does not necessarily impose a limitation on the scope of the invention unless otherwise specifically recited in the claims. No language in the specification should be construed as indicating that any non-claimed element is essential to the practice of the invention.

### EXAMPLE 1

### CD24 as a Biomarker in Myeloma-Initiating Cells

Tumor-initiating cells (TICs) or cancer stem cells (CSCs) were originally documented and described in leukemia as a rare population of cells with limitless self-renewal capabilities^{1,2}. Recently, TICs have been identified in a growing number of solid tumors³⁻¹⁶. A common feature of tumor-initiating cells is their increased resistance to chemo- and radiotherapy¹⁷⁻¹⁹. Treatment failure in cancers, including multiple myeloma (MM), is mostly likely due to persistence a minor population of TICs, which are non-cycling or low-cycling and very drug-resistant tumor cells. MM is a malignant disease, characterized by an excess of clonotypic plasma cells in the bone marrow (BM)²⁰. Leung-Hagesteijn and colleagues described that negative spliced X-box binding protein 1 message (XBP1s⁻) subpopulations of tumor cells are precursors of MM cells and are resistant to bortezomib²¹. Tanno et al. demonstrated that GDF15 enhances the tumor-initiating and self-renewal potential of MM cells and increased serum GDF15 was associated with an inferior outcome in MM patients²². Side population (SP) cells from different MM cell lines generated more colonies when compared with mature plasma cells and this SP lacked correlation with CD138 expression^{23,24}. Typically, plasma cells actively secrete intact monoclonal immunoglobulin (IgG, IgA, IgD, or IgE) and/or free monoclonal κ or λ or X light chains ²⁵⁻²⁸. The majority of patients ultimately relapse with a drug-resistant disease, presumably derived from the persistence of a small subset of MM TICs. More recently, a drug-resistant subpopulation of memory B cell-like cells with the CD138⁻/CD19⁺/CD27⁺ or light chain-restricted (LCR) CD19⁺ phenotype in MM was identified and termed MM TICs²⁹⁻³¹. However, a universally accepted TIC phenotype has not yet been established, which has hampered the acceptance of the MM TIC concept.

The human cell surface antigen CD24 is a sialoglycoprotein localized in membrane lipid raft domains³². CD24, a heat-stable antigen, was used as a marker to differentiate hematopoietic cells and neuronal cells and B lymphocytes^{33,34}. It is also expressed on normal monocytes, granulocytes, red blood cells, platelets and activated T lymphocytes³⁴. A CD24 knock-out mouse had no other functional defect but B-lymphocyte development, indicating that CD24 is involved in the proliferation and maturation of pro B-lymphocytes. Recently, many studies indicate that CD24 is expressed and has been recognized as a cancer stem cell marker in multiple cancers^{33,35-41}. For example, CD24 over-expression was observed in ovarian cancer, breast cancer, small cell lung cancer, prostatic cancer, pancreatic cancer, rectal cancer, bladder cancer and cholangiocarcinoma, and its expression is associated with poor prognosis⁴². CD24 expression seems to be a key factor in metastasis. CD24⁺ cells can interact more easily than CD24⁻ cells with P-selectin detected in the activated platelets in blood to form a blood clot. Cancer cells migrate far through blood flow by being carried on the generated blood clot. CD24 can easily adhere to endothelial cells of a target organ to induce metastasis.

In this study, CD24⁺ MM cells were identified have potential TIC features from a systemic analysis of primary MM samples and MM cell lines using GEP. The mechanisms how CD24 maintains the feature of self-renewal and drug resistance in MM was determined.

### RESULTS

Persistence of Tumor-Initiating Cells in Multiple Myeloma by Analyzing Complete Remission and Relapsed Primary Myeloma Samples

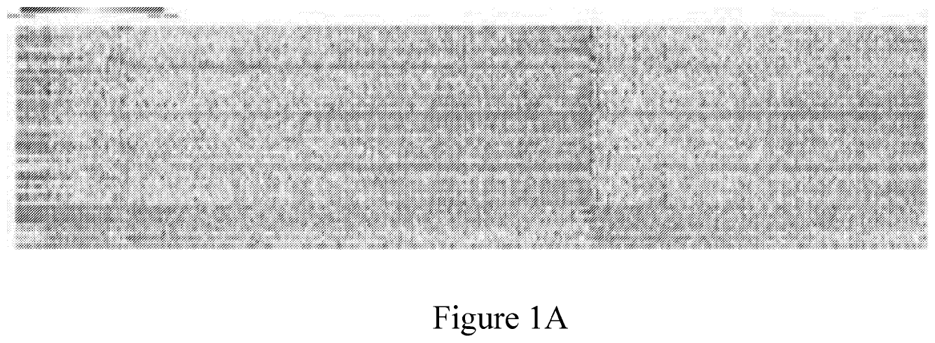

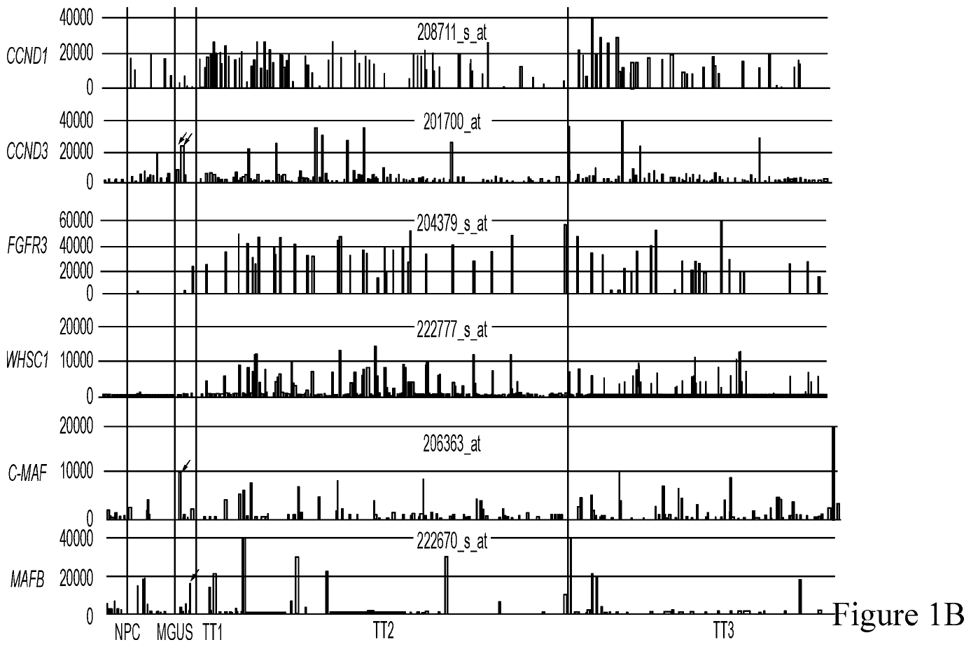

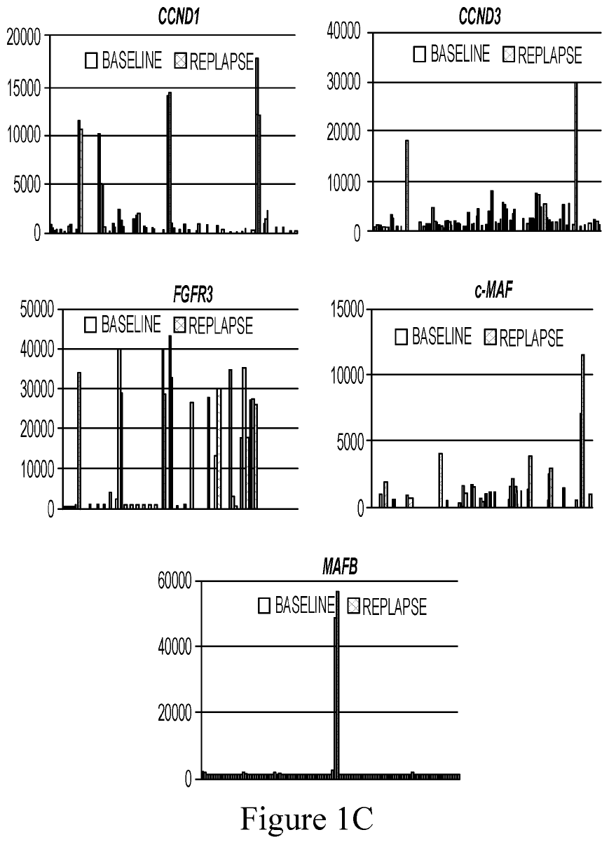

Because reciprocal translocations of t(4;14), t(11;14), t(6;14), t(14;16), and t(14;20) are the tumor initiation factors in more than 40% newly diagnosed MM patients, we investigated whether these initiators exist in MM samples after chemotherapy and Autologous Stem Cell Transplant (ASCT) by analyzing samples collected in Total Therapy 1 (TT1) samples who had MM surviving more than 10 years after initiation of TT1 using gene expression profiling (GEP). Firstly, a heatmap in FIG. 1A presents the expression of 52 MGUS genes in normal plasma cells (n=22), MGUS (n=44), and MM (TT1, n=20; TT2, 351; and TT3, n=206). The TT1 MM samples show a similar expression pattern to MGUS, which distinguishes from most of newly diagnosed MM samples derived from TT2 and TT3 trials. The reciprocal translocations in MM are nonrandom chromosomal fusions driving high expression (spike) levels of the respective partner genes, such as spike CCND1 in t(11;14), CCND3 in t(6;14), FGFR3 and MMSET in t(4;14), c-MAF in t(14;16), and MAFB in t(14;20). As shown in the FIG. 1B, a total of four spikes including two CCND3, one c-MAF, and one MAFB were identified from the 20 TT1 MM samples, indicating that the translocation clone persists in MM patients with long-lasting complete remission (CR; >10 years). We then analyzed these spike genes' expression in 51 paired samples collected at diagnosis and at early relapse from the same patient in the TT2 cohort. These patients were responded to the treatment on the basis of Thalidomide and ASCT and had partial or complete remission. The spike expression of CCND1, CCND3, FGFR3, c-MAF, and MAFB were identified from 18 of 20 in both samples at diagnosis and at relapse, only one loss of FGFR3 spike was found at diagnosis, while another loss of CCND3 spike was found at relapse (FIG. 1C). These observations suggest the persistence of a cancer cell population, namely tumor-initiating cell (TIC), in MM.

Identify Cell Surface Protein CD24 as a Biomarker of Tumor-Initiating Cells in Myeloma

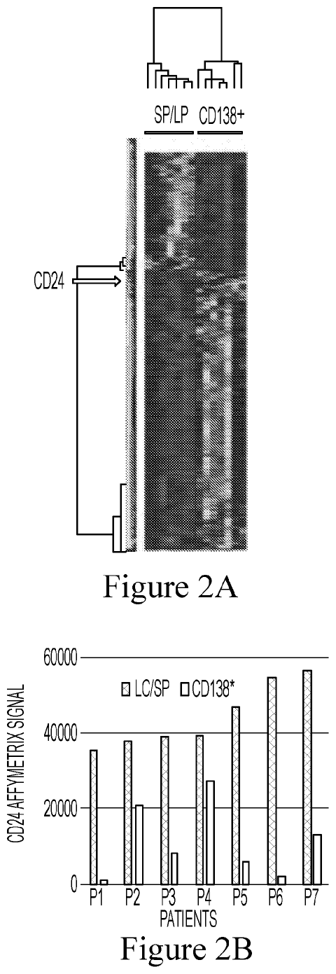

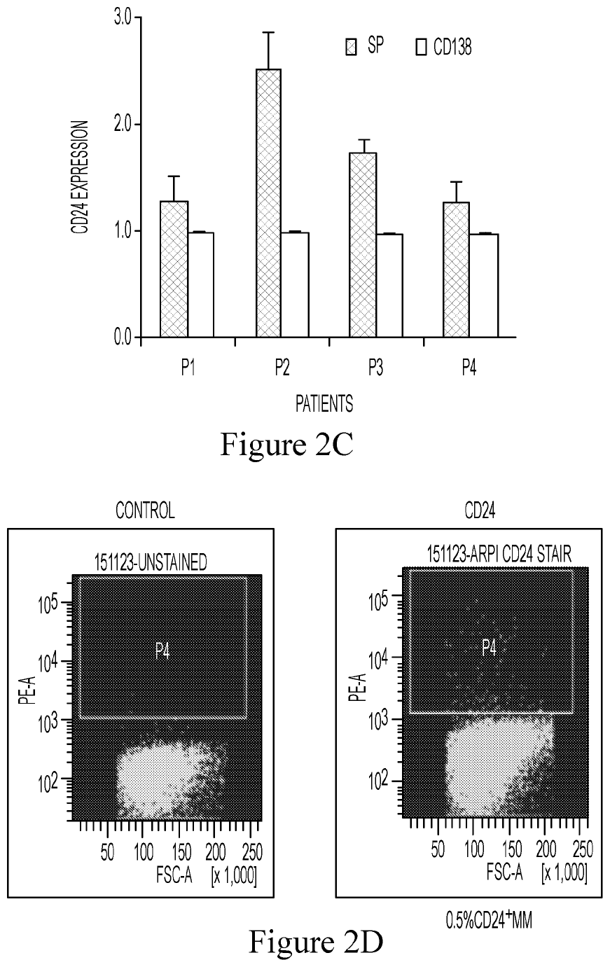

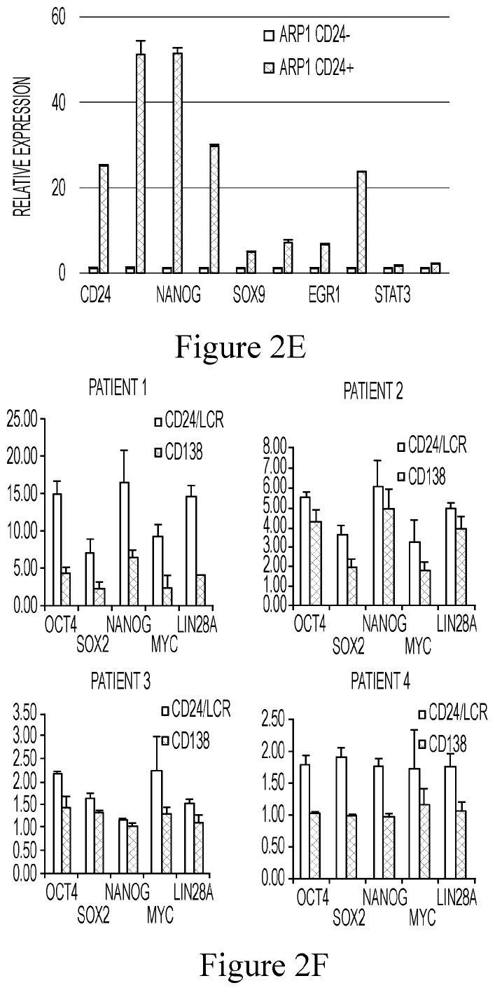

Since none reliable cell surface markers have been identified for MM TICs and side population (SP) as a surrogate of TIC marker has been widely accepted in cancer including MM, we isolated MM initiating cells with MM clonotypic marker either k⁺ or λ⁺ light chain plus the side-population (LC/SP), from seven clinical samples. We then performed Affymetrix microarrays on paired LC/SP and the bulk MM cells (CD138⁺) on these 14 samples. More than 1000 genes were identified significantly differentially expressed between these two groups (FIG. 2A). We are particularly interested in genes encoded cell surface proteins and found that IL8A, TNFRSF10C, CD24, and CEACAM1 genes were top upregulated in the LC/SP MM cells compared to CD138⁺ MM cells (FIG. 2B). We then performed qRT-PCR to confirm the expression of these four genes and the expression of iPS/ES genes, such as OCT4, NANOG, and SOX2, between LC/SP and CD138+ MM cells from another four primary MM samples (data not shown). Only CD24 showed a consistent result with the microarray data, which CD24 was significantly higher in LC/SP than in CD138+ bulk MM cells. Importantly, the expression of iPS/ES genes, such as OCT4, NANOG, and SOX2, was also significantly upregulated in LC/SP MM cells compared to CD138+ MM cells. However, we could not verify the microarray data for the other three genes, i.e., IL8A, TNFRSF10C, and CEACAM1, by qRT-PCR (data not shown). Flow cytometry was further used to detect CD24 in multiple MM cell lines (ARP1, OCI-MY5, JJN3, KMS28PE and H929), we identified CD24+ MM cells as a distinct rare population (0.4˜3.1%; FIG. 2D). CD24⁺ MM cells were sorted out from the above five MM cell lines, the expression of CD24 and OCT4, NANOG, and SOX2 was evaluated by qRT-PCR. The expression of CD24 and iPS/ES genes was significantly higher in CD24⁺ MM cells than in CD24⁻ MM cells (FIG. 2E). The expression of iPS/ES genes was compared between CD24+/LC MM cells with the CD138+ MM cells by qRT-PCR (FIG. 2F). Therefore, we focus on studying CD24 as a putative TIC biomarker in this study.

CD24 Myeloma Cells are Enriched After Chemotherapy and in Complete Remission of Myeloma

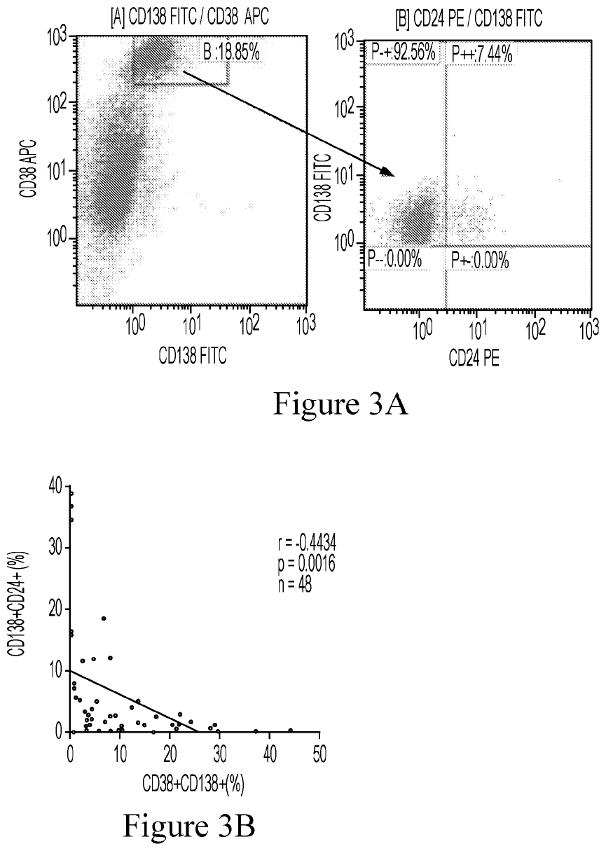

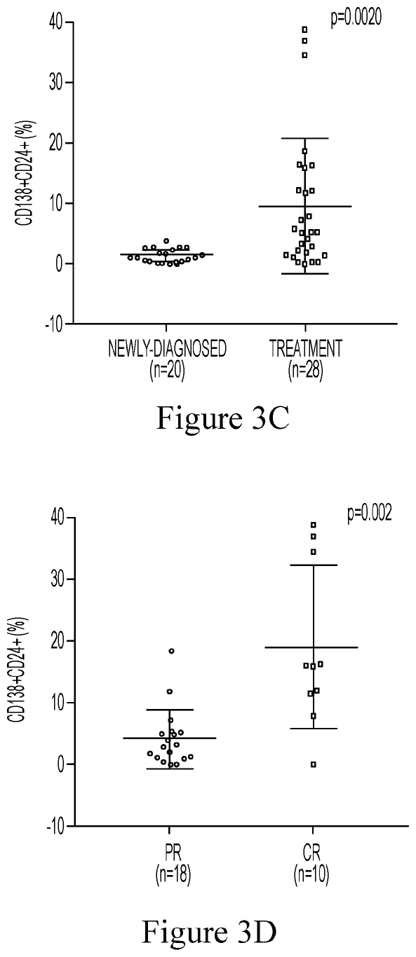

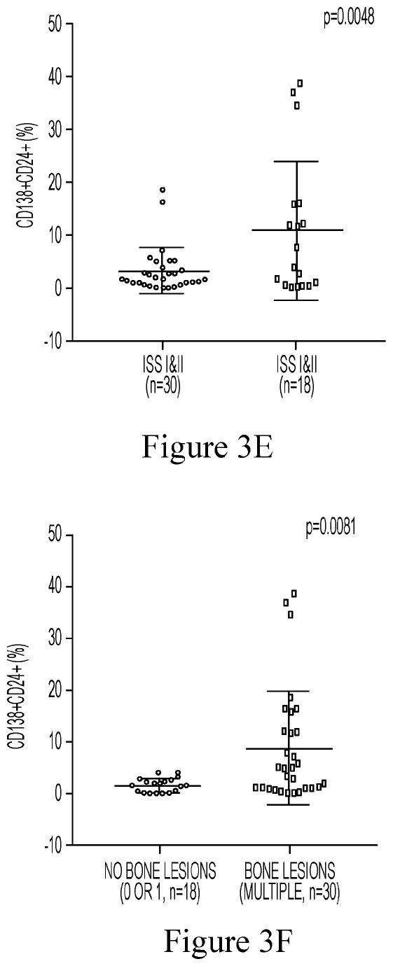

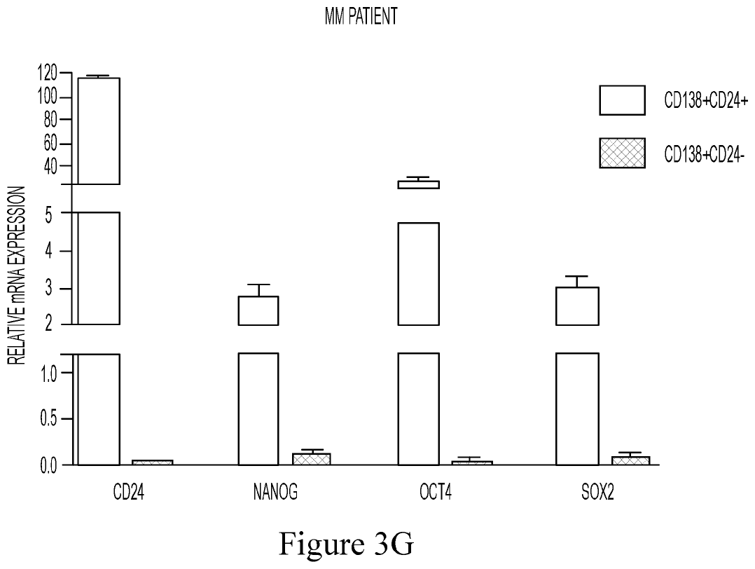

To confirm the presence of MM CD24⁺ subpopulation in primary MM patients, we first analyzed 11 patients' samples by flow cytometry (FACS) using the panel of plasma cell markers (CD138, CD38, k⁺ or λ⁺ light chain, and CD56), and B cell makers (CD45, CD19), as well as CD24 antibodies. We discovered that CD24⁺ subpopulation was present in MM cells defined by plasma cell markers from eight of the 11 different MM samples. We then expand the FACS analysis in another 48 MM patients using CD138, CD38 and CD24 antibodies. As shown in the FIG. 3A and Table 1, a subset of primary MM cells was CD24-positive in CD138⁺CD38⁺ MM cells detected by flow cytometry. We found a negative correlation between CD138⁺CD38⁺ percentage with CD138⁺/CD24⁺ percentage (FIG. 3B; r=−0.4434, p=0.0016). We hypothesized that current intensive treatments ineffectively eliminate the small population of TICs while effectively eliminating the bulk of the “more chemo-sensitive” MM cells and that this allows subsequent MM relapse and treatment failure. Consistently with our hypothesis, the mean percentage of CD138⁺CD24⁺ MM cells was 1.1% in the 20 newly diagnosed MM samples ranged from 0% to 3.8%, while it was 8% in the 28 samples after treatment (FIG. 3C; p=0.0020), the proportion of CD138⁺CD24⁺ MM cells were also significantly increased in patients with complete remission (CR) compared to those in partial remission (PR) (FIG. 3D; p=0.0002). In addition, increased CD138⁺CD24⁺ population was identified in advanced disease (FIG. 3E; ISS III versus ISS I & II; p=0.0048) and patients with more bone lytic lesions detected by x-Ray (Figure F; p=0.0081). The expression of iPS/ES genes were also analyzed in clinical samples, FIG. 3G showed that the expression of CD24, NANOG, OCT4, and SOX2 were significantly higher in CD138⁺/CD24⁺ MM cells compared to CD138⁺/CD24⁻ MM cells.

TABLE 1
the correlations of clinical characteristics with CD24 in 48 MM patients

| Clinical index | | | | | p value |
|---|---|---|---|---|---|
| Gender | Male | (29) | Female | (19) | 0.526 |
| Age (years) | <65 | (29) | ≥65 | (19) | 0.375 |
| lgA isotype | lgA | (17) | Others | (31) | 0.231 |
| ISS stages | I & II | (30) | III | (18) | 0.004 |
| DS stages | I & II | (15) | III | (33) | 0.041 |
| DS subgroups | A | (38) | B | (10) | 0.029 |
| Laboratory examination | | | | | |
| sFLC K/L | 0.26-1.65 | (16) | without | (32) | 0.001 |
| M protein (g/L) | <0.2 | (24) | ≥0.2 | (24) | 0.003 |
| sCr (μmol/L) | <176.8 | (38) | ≥176.8 | (10) | 0.029 |
| β2-MG (mg/L) | <4 | (20) | ≥4 | (28) | 0.032 |
| CRP (mg/L) | <4 | (28) | ≥4 | (20) | 0.439 |
| ESR (mm/H) | <100 | (28) | ≥100 | (20) | 0.633 |
| HB (g/L) | <100 | (30) | ≥100 | (18) | 0.914 |
| ALB (g/L) | <35 | (14) | ≥35 | (34) | 0.149 |
| LDH (U/L) | <190 | (26) | ≥190 | (22) | 0.834 |

p < 0.05 was considered to reflect statistical significance.
Abbreviations:
sCr, Serum creatinine;
CRP, C-reactive protein;
ESR, Erythrocyte sedimentation;
ALB, Serum Albumin;
β2-MG, β2-Microglobulin;
LDH, Lactate Dehydrogenase

CD24 Myeloma Cells Show Strong Clonogencity and Tumorigenesis

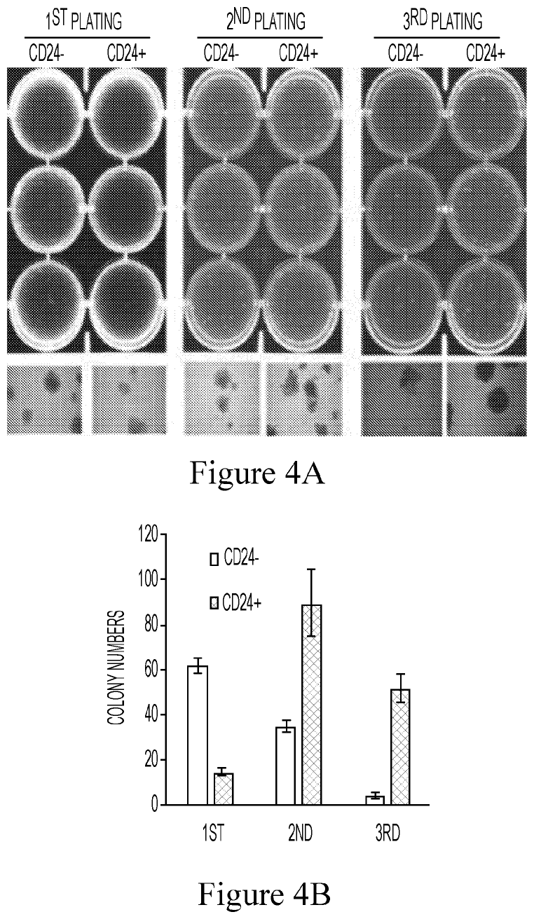

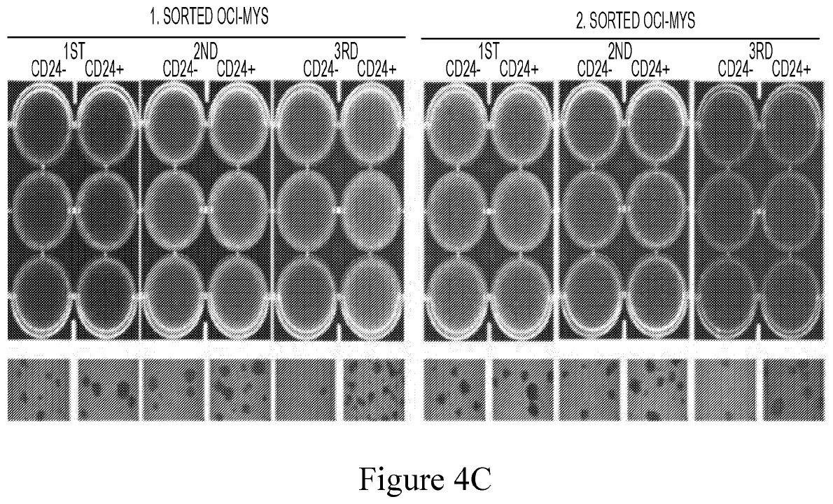

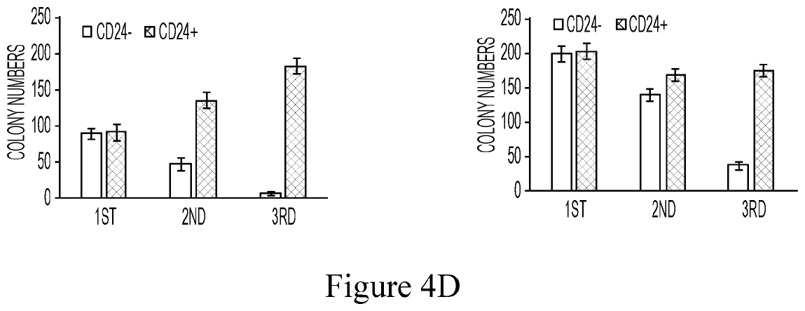

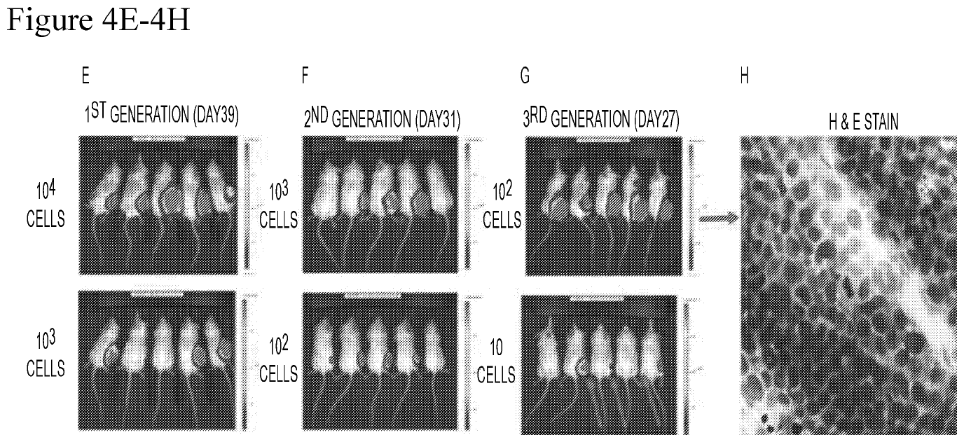

To evaluate the possibility that CD24⁺ MM cells show higher clonogenicity than bulk MM cells, we isolated CD24⁺ and CD24⁻ cells from ARP1 and OCI-MY5 cell lines and examined each subpopulation for colony formation in methylcellulose. To examine self-renewal potential, colonies were serially replated. Initially, CD24⁺ cells from both cell lines yielded less colonies in the first plating, but CD24⁺ cells underwent significantly greater clonogenic expansion than CD24⁻ cells in the 2^{nd} replating and finally in the third plating, CD24⁺ generated much more colonies than CD24⁻ cells, suggesting that CD24⁺ cells hold for the long-term self-renewal feature of TICs (FIGS. 4A & 4B). We also isolated CD24⁺ and CD24⁻ cells from secondary and tertiary xenografts in mice from ARP1 and OCI-My5 cells, then perform colony-formation assay. Similar results were observed that CD24⁺ cells generated much more colonies than CD24⁻ cells during serial replating (data not shown), suggesting that CD24⁺ cells possess greater self-renewal capacity. Similar results were observed from the OCI-MY5 cell line (FIGS. 4C & 4D).

We then investigated if CD24⁺ MM cells show increased tumorigenic property compared to the CD24⁻ MM cells in mice. Luciferase ARP1 (ARP1-Luc) and luciferase OCI-MY5 (OCI-MY5-Luc) cell lines were sorted out CD24⁺ and CD24⁻ populations. CD24⁺ and CD24⁻ MM cells from both cell lines were injected subcutaneously into the right and left flanks of NOD-Rag/null gamma (NSG) mice, respectively. Tumor development and growth were monitored by bioluminescence weekly. In the 1^{st} generation, 10,000 CD24+ ARP1 cells formed tumors in five out five mice after 39 days' injection, while 10,000 CD24⁻ ARP1 cells formed tumor in one out five mice. With injection of 1000 cells per mouse, CD24⁺ ARP1 cells generated tumors in four out five mice, while no tumor was detected in the left side, which received 1000 CD24⁻ ARP1 cells. To further assess the self-renewal capacity of CD24⁺ MM cells, serial transplantations were performed. The corresponding tumors were excised from the primary recipients, dissociated into a single-cell suspension, resorted into CD24⁺ and CD24⁻ MM cells, and then re-injected into secondary and tertiary recipients (FIGS. 4E˜4G). One thousand or one hundred CD24⁺, but not CD24⁻, ARP1 cells were able to develop tumors in four of five mice after 4 weeks' injection in the 2^{nd} transplantation (FIG. 4F, 2^{nd} passage). In the 3^{rd} transplantation, 100 CD24⁺ MM cells developed tumors in all five mice, and even ten CD24⁺ MM cells developed tumors from three of five mice in 27 days (FIG. 4G, 3^{rd} passage). The tumor cells were confirmed by the H & E staining (FIG. 4H).

CD24 Myeloma Cells are Resistant to Multiple Drugs

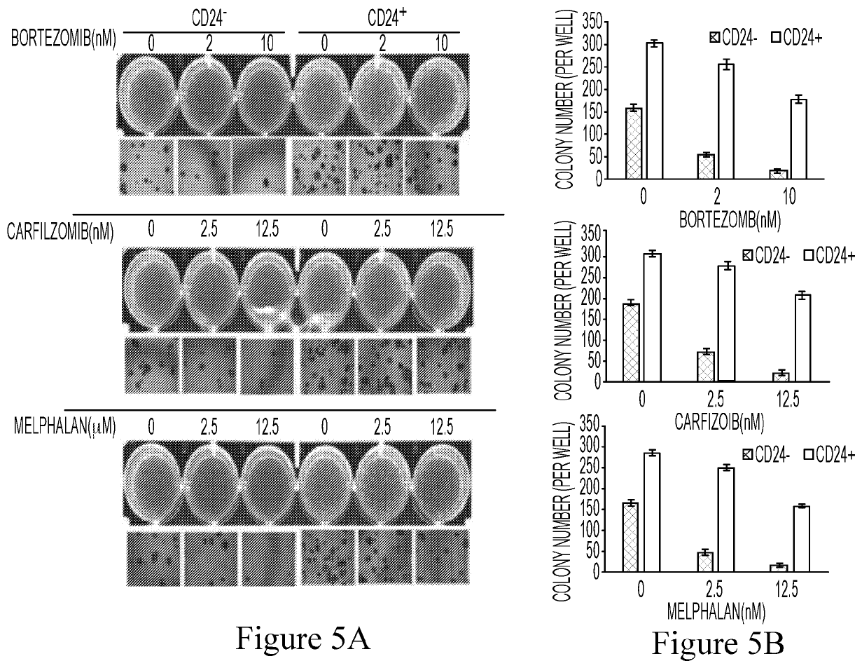

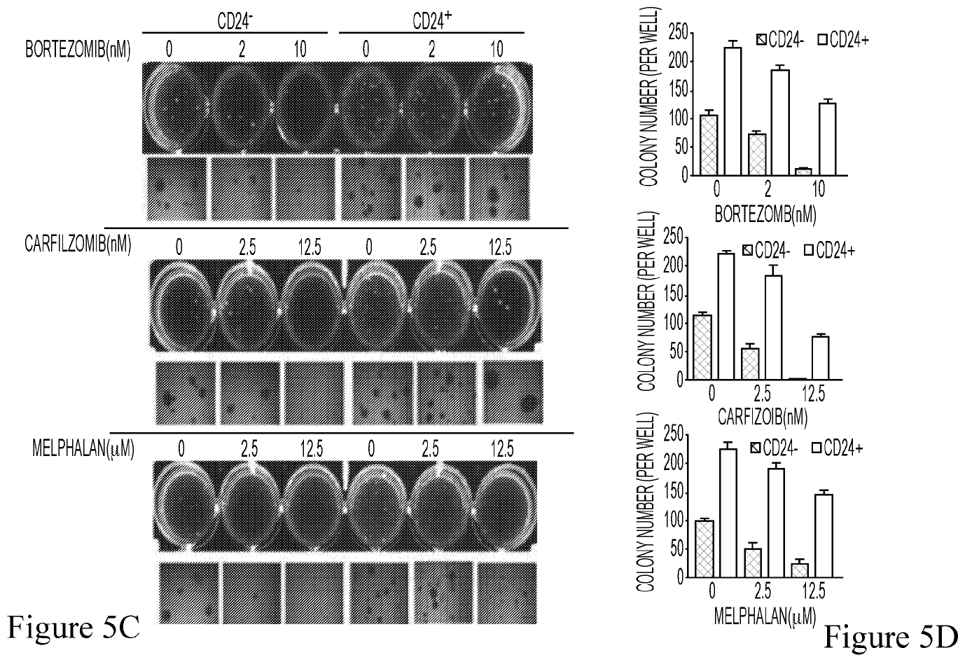

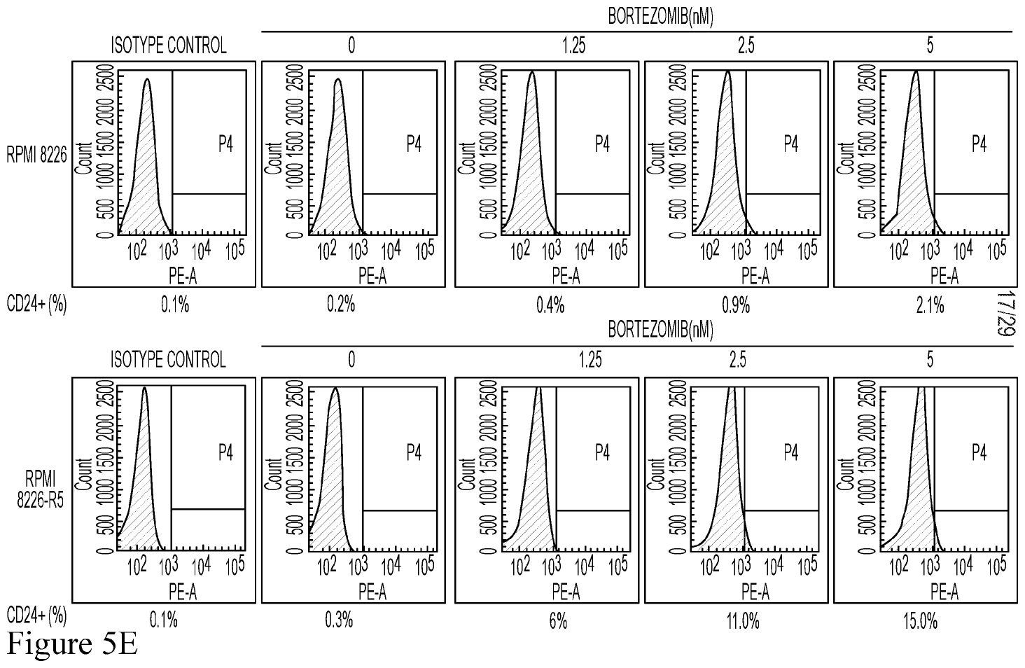

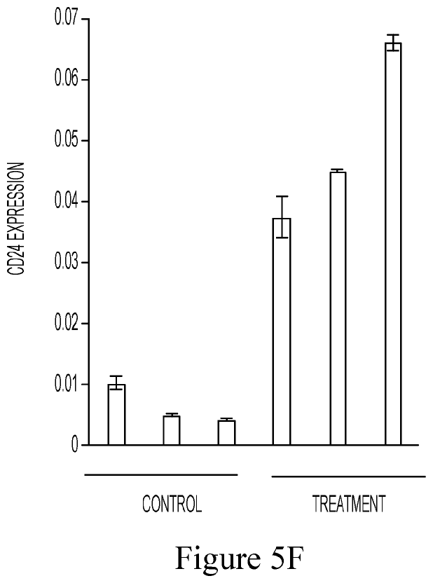

Drug resistance is a critical characteristic of TICs⁴³. To examine whether CD24⁺ cells show higher resistance to chemotherapeutic drugs, the soft agar clonogenic formation assay was employed to examine the response to multiple drugs. The 2^{nd} passage cells were used for this test, since our study presented in Figure x showed only the 2^{nd} and 3^{rd} passages of CD24⁺ MM cells had more colonies than those of CD24⁻ MM cells. The ARP1 cells from the 1^{st} passage were collected and re-plated in methylcellulose dishes. CD24⁺ and CD24⁻ ARP1 MM cells in the 2^{nd} passage were treated with bortezomib (2 and 10 nM) or carfilzomib (2.5 and 12.5 nM) or melphalan (2.5 and 12.5 μM) for 2 weeks. CD24⁺ ARP1 (FIGS. 5A & 5B) or OCI-MY5 (FIGS. 5C & 5D) MM cells were more pronounced resistant (>2.5-fold) than CD24⁻ ARP1 or OCI-MY5 MM cells. To directly confirm whether CD24⁺ population are enriched upon chemotherapy, we treated RPMI-8226, RPMI-8226-R5 (a drug resistant cell line) cells with bortezmib or melphalan in vitro for 72 h (data not shown). Flow cytometry indicated that CD24+ population was dramatically increased in the chemoresistant residual cells in the above cell lines (FIG. 5E, 2˜100 folds). Accordingly, the expression of CD24 was upregulated in the survival cells compared to the untreated MM cells (FIG. 5F).

STAT3 is the Major Signaling Pathway of Tumor-Initiating Cells in Myeloma

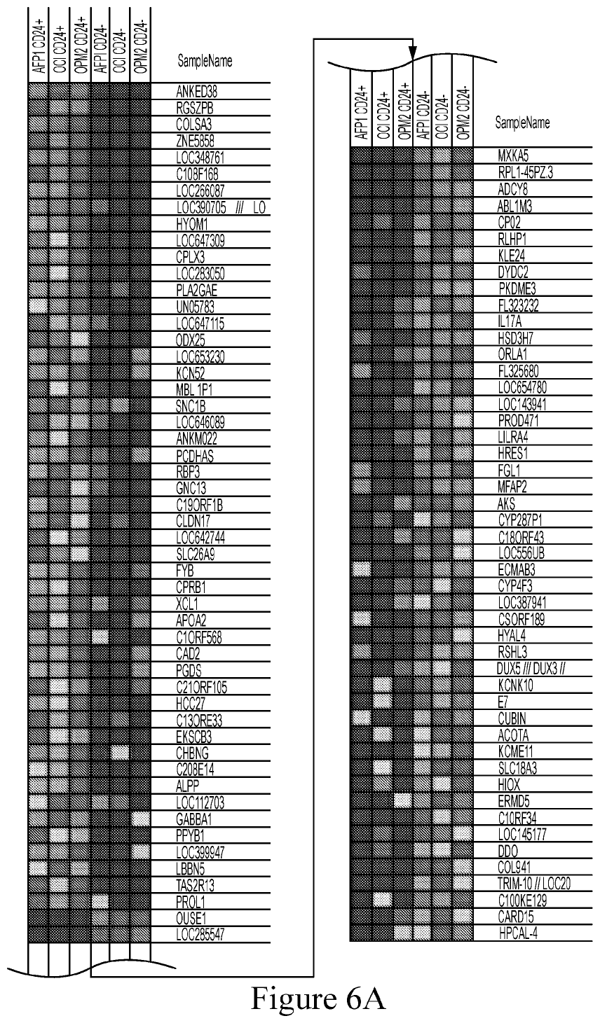

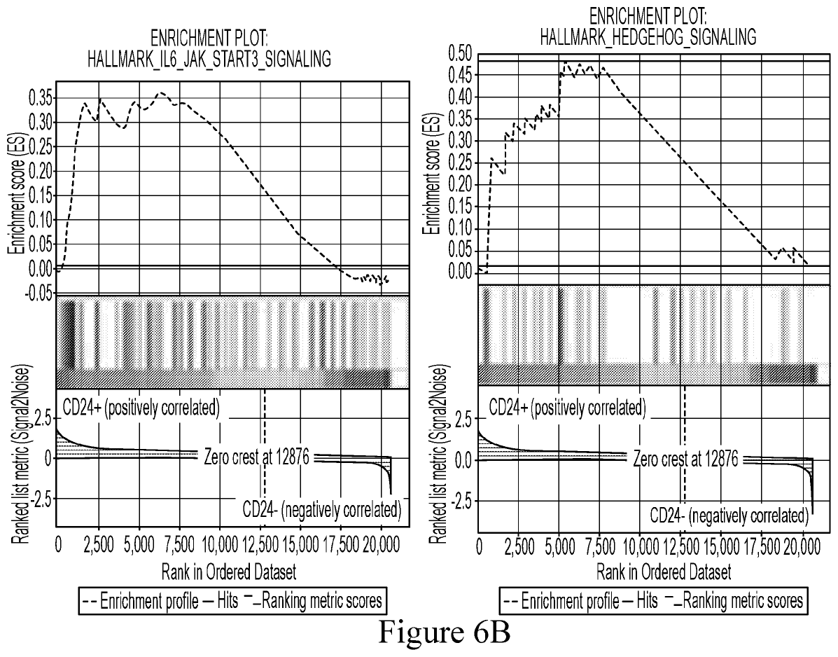

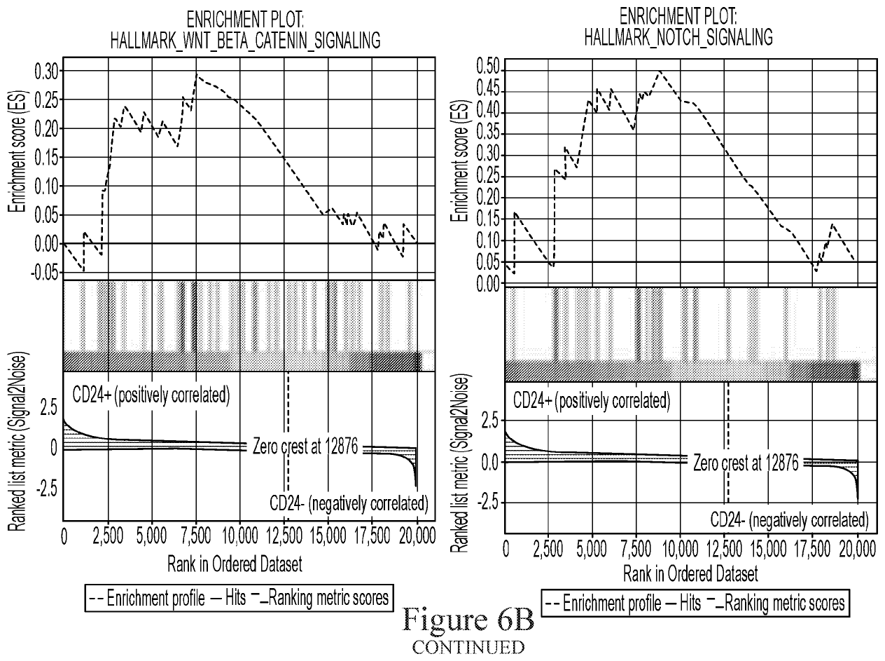

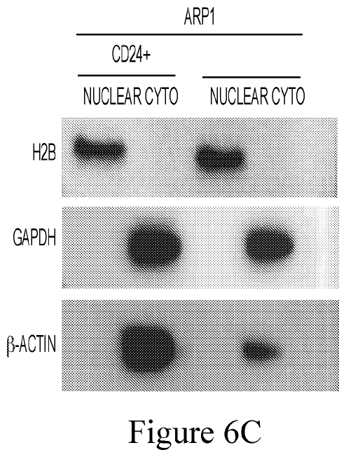

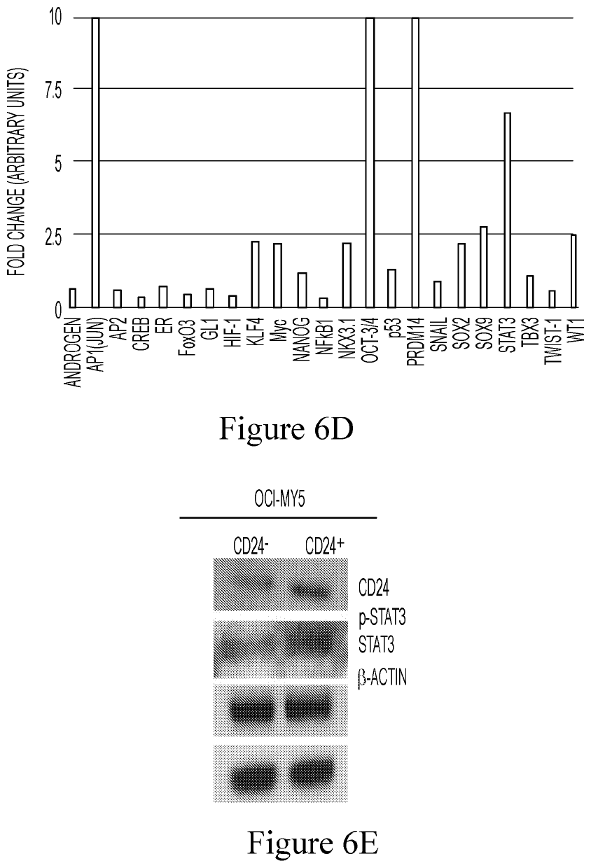

To identify CD24 signaling pathways for maintaining TIC features, GEPs were performed from CD24⁺ and CD24⁻ MM cells sorted from ARP1, OCI-MY5 and OPM2 lines. More than 200 genes were distinctly expressed between CD24⁺ and CD24⁻ MM cells (p <0.01). FIG. 6A showed top 50 upregulated or 50 downregulated genes in CD24⁺ MM cells. We analyzed the CD24 signaling pathways using Gene Set Enrichment Analysis (GSEA). Of the 25 top positively correlated pathways, the JAK2-STAT3 pathway was the most significantly enriched at nominal p-value <1% in CD24⁺ MM cells (FIG. 6B). Because STAT3 is a transcription factor (TF), we further screened a transcription factor profiling array including 23 cancer stem cell transcription factors (TFs). CD24⁺ and CD24⁻ populations from ARP1 cells were sorted out and nuclear proteins were isolated for the analysis with activity of transcription factors plated on the cancer stem cell TF arrays. After confirmation of successful nuclear extracts (FIG. 6C), the nuclear extracts were mixed with a biotin-labeled pool of DNA probe mix that correspond specifically to TF response elements. Following the protocol for incubation, hybridization and elution, etc, the activity signals of each TF were detected with a Streptavidin-HRP and HRP substrate, chemiluminescence was measured by a plate reader. We found that the activities of AP1, OCT 3/4, PRDM14, and STAT3 were significantly higher in the CD24⁺ population compared to the CD24⁻ population (FIG. 6D). To determine whether STAT3 activity was truly activated in CD24⁺ MM cell, western blots were also probed on cell lyses of CD24⁺ and CD24⁻ MM cells sorted from OCI-MY5 with both phosphorylated STAT3 and total STAT3 antibodies. FIG. 6E showed that p-STAT3, not the total STAT3 protein, was significantly increased in CD24⁺ MM cells compared to CD24⁻ MM cells. This was further confirmed in CD24-overexpressing OCI-MY5 MM cells (data not shown). Together, STAT3 signaling was the most activated pathway in CD24⁺ MM cells compared to CD24⁻ MM cells.

Targeting STAT3 Prevents Tumor-Initiating Cell Growth

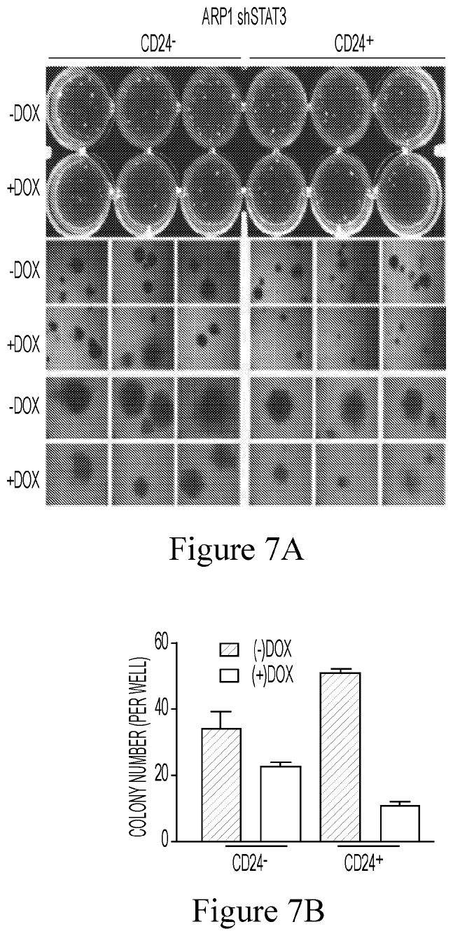

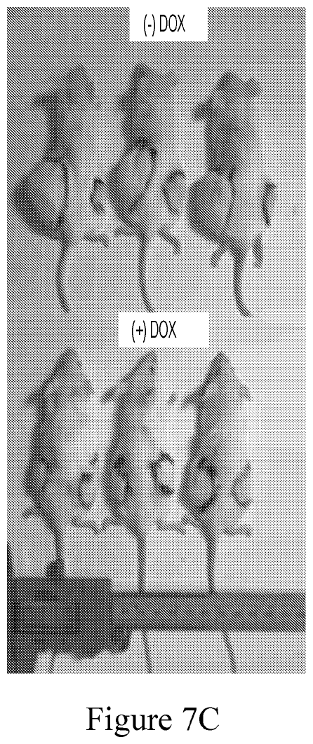

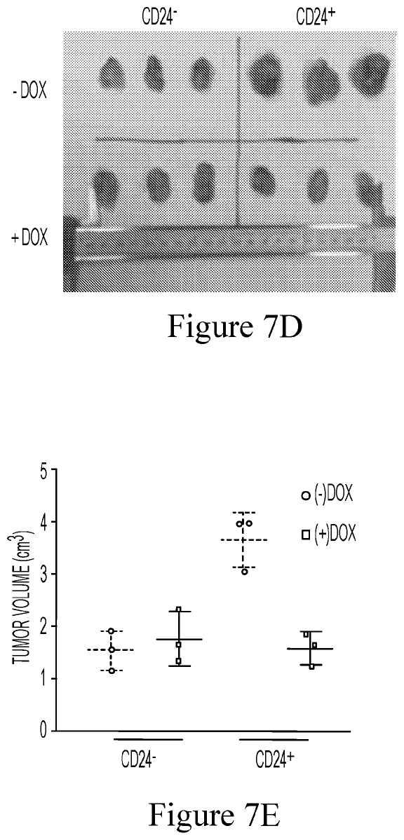

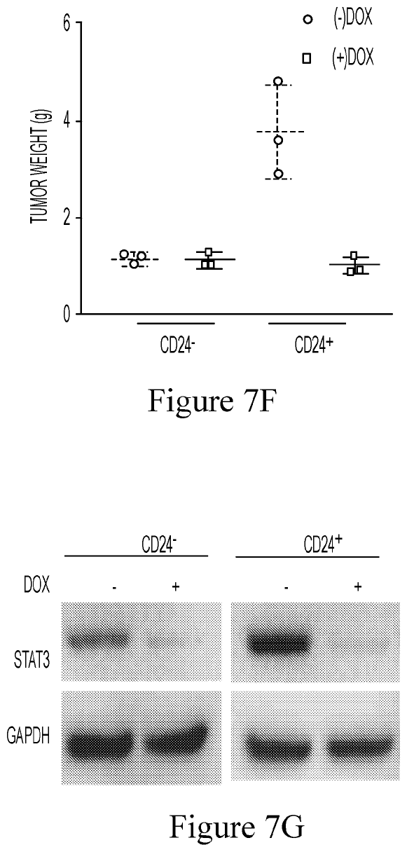

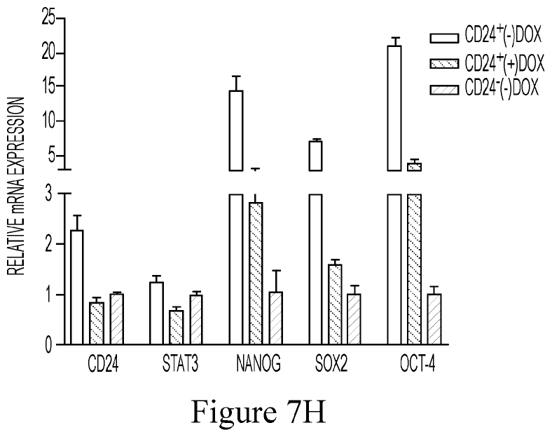

To determine whether STAT3 mediates CD24 TIC features, we firstly evaluated whether knockdown of STAT3 could block CD24 clonogenicity. We have generated inducible STAT3 shRNA in ARP1 MM cells. The 2^{nd} passage of CD24⁺ and CD24⁻ ARP1 cells were sorted and plated for colony formation in soft-agar plates. Doxycyline was added in 24 h after plating. As expected, CD24⁺ ARP1 cells had more colonies than CD24⁻ ARP1, and silencing STAT3 decreased colonies in both CD24⁺ and CD24⁻ MM cells (FIG. 7A). However, the colony number decreased more significantly in CD24⁺ ARP1 than CD24⁻ ARP1 cells (FIGS. 7A & 7B).

The effect of inhibition of STAT3 in CD24⁺ MM cells was also evaluated in vivo using xenograft model. We have shown that injection of 1,000 CD24⁺ MM in the 2^{nd} transplantation resulted in tumor development in one month. To determine whether STAT3 is essential for myelomagenesis in vivo, 1,000 CD24⁻ and 1,000 CD24⁺ ARP1 cells engineered STAT3-shRNAs were injected into right and left flank of every NOD-Rag1^{null} mouse respectively. Doxycycline was added to the drinking water to knock down STAT3 expression in half of the mice (n=3) after injection of MM cells. Mice were euthanized after 33 days induction of doxycycline and tumors were excised for analyses. Inhibition of STAT3 had a strong anti-tumor effect in CD24⁺ MM cells, while it did not show significance in CD24⁻ MM cells from the mouse pictures (FIG. 7C) and harvested tumors photographed (FIG. 7D). Quantifications of tumor volume and weight showed consistently inhibition of tumor growth in CD24⁺ MM cells (FIGS. 7E & 7F). Tumor cells were also isolated for western blotting, STAT3 levels were depleted in tumor cells silenced STAT3 in both CD24⁺ and CD24⁻ MM cells (FIG. 7G). The iPS/ES genes' expression was determined by qRT-PCR and STAT3 shRNA significantly inhibited the expression of NANOG, OCT4, and SOX2 in CD24⁺ ARP1 cells (FIG. 7H). Together, we show that CD24 activates iPS/ES genes signaling via STAT3 and targeting STAT3 prevents CD24 functions of tumorigenesis.

Discussion

One major clinical observation of MM TICs is that we have shown that gene expression profiles (GEP) remain abnormal in MM patients with long-lasting complete remission (CR) (>10 years)⁴⁴, suggesting the persistence of a cancer cell population with low proliferative capacity and limited sensitivity to our most intensive therapies. The phenotype of MM TICs has remained unclear and controversial. Different groups have reported on the presumed identity of MM TICs. Memory B cell-like cells with the CD138⁻/CD19⁺/CD27⁺ or light chain-restricted (LCR) CD19⁺ phenotype in MM was identified and termed MM TICs²⁹⁻³¹. Paradoxically, mature CD138⁺ MM cell de-differentiation from a CD34⁺/CD138⁺/B7⁻/H1⁺ subpopulation to MMSCs was also described⁴⁵; in addition, CD38⁺⁺/CD45⁻ plasma cells proliferate successfully within an engrafted human fetal bone using the SCID-hu mouse model^{46,47}. Consistently, Weissman group demonstrate that fully differentiated plasma cells (CD138⁺/CD38⁺/CD19⁻/CD45^{low/-}) enrich for long-lived and tumor-initiating cells whereas B cells or plasmablasts do not⁴⁸. To identify phenotypic cell surface markers of MM TIC, we performed GEPs, including all human genomic sequences, on MM cells obtained from seven MM patients with paired side population (a surrogate of stem cell marker) plus a MM specific marker, the clonotypic light chain-restricted (LC/SP) versus the bulk MM cells (CD138⁺). The cell surface protein CD24 was found to be significantly upregulated in the LC/SP MM cells compared to the bulk CD138⁺ MM cells.

To determine whether CD24 is a potential marker of MM TICs, we isolated CD24⁺ and CD24⁻ subpopulations from MM cell lines. We have shown that CD24⁺ MM cells have increased clonogenic potential, drug resistance in vitro and tumorigenesis in vivo after injecting only 10 cells from MM cell lines. It is known that CD24 is absent on normal plasma cells⁴⁹⁻⁵¹. However, we discovered that the presence of CD24⁺MM cells is highly variable in primary MM samples. To confirm the presence of MM CD24⁺ subpopulation in MM patients, we analyzed 11 patients' samples by flow cytometry using the panel of plasma cell markers (CD138, CD38, k⁺ or λ⁺ light chain and CD56), and B cell makers (CD45, CD19), as well as CD24 antibodies. We discovered that CD24⁺ subpopulation was present in MM cells defined by plasma cell markers from eight of the 11 different MM samples. We further expanded analyses in another 48 primary MM samples using flow cytometry. We determined that the subpopulation of CD24⁺ MM cells enriched in patients after chemotherapy, in complete remission, advanced stage, and with more bone lytic lesions. These data support that an increase of CD24⁺ MM cells is a reliable predictor of disease progression in MM.

The property of self-renewal is shared by pluripotent iPS/ES and cancer stem cells. The core pluripotency factors NANOG, OCT4 (also known as POU5F1) and SOX2 collaborate with the accessory proteins LIN28, MYC and KLF4 to form a self-reinforcing regulatory network that enables the stable expression of self-renewal factors and the repression of genes that promote differentiation⁵²⁻⁵⁴. These transcription factors are also sufficient to reprogram terminally differentiated tissues such as fibroblasts and B cells into induced pluripotent stem cells^{55,56}. Our studies from both MM cell lines and primary MM samples showed that CD24⁺ cells had significantly higher expression of iPS/ES genes, i.e. NANOG, OCT4, and SOX2, etc. To further determine how CD24 maintains the TIC signaling, we compared microarrays between CD24+ and CD24− MM cells. The GSEA identified that STAT3 signaling is the most activated pathway in CD24+ MM cells. This was further supported by screening a stem cell TF array that includes 23 cancer stem cell transcription factors (TFs). We found that STAT3 was one of four significantly activated proteins from nuclear extracts of CD24⁺ MM cells compared to CD24⁻ MM cells. It is interesting to note that knockdown of STAT3 in CD24+ MM cells decreased the expression of iPS/ES genes and abolished the colonegencity and tumorigenecity. Therefore, we conclude that CD24, via STAT3 signaling, maintains the myeloma-initiating cell features of self-renewal and drug resistance.

Other stem cell related signaling pathways, such as Wnt, Hedgehog and Notch, were activated in CD138⁻ MM cells compared to bulk MM cells^{29,30,57} and also in CD24⁺ MM cells from this study by GEP. In summary, our study is innovative and the first one shows that CD24⁺ is a key marker for MM TICs as it is in other cancer stem cells. Our results add invaluable information about new therapeutic approaches for drug resistant MM cells and impact the clinical outcome of MM.

Materials and Methods

Gene Expression Profiling

The data of gene expression profile (GEP) were collected from a publicly available website that include 22 normal plasma cells from normal donors, 44 MGUS, 20 MM samples after long-term remission in the Total Therapy 1 (TT1) clinical trial, 351 and 206 newly diagnosed patients with MM who participated in the TT2 and TT3 clinical trials respectively. In addition, 268 newly diagnosed samples from the HOVON-65 with GEP and clinical outcome were also included in this study.

Side-population/light chain⁺ cells. CD138⁺ MM cells, CD138⁺/CD24⁺, or CD138⁺/CD24− MM were sorted out from primary MM samples, and CD24⁺ or CD24⁻ MM cells were also sorted out by flow cytometry. Cells were washed then resuspended in phosphate buffered saline (PBS) and sorted on a FACS LSR (Becton Dickinson) or analyzed by flow cytometry. Importantly, viable cells were stained with Hoechst 33258 (1 μg/mL) (Invitrogen).

GEP and data analysis, using the Affyrnetrix U133Plus2.0 microarray, were performed.

Cell Lines and Cell Culture

Human MM cell lines ARP1, OCI-My5, OPM2, JJN3, H929 and KMS28PE cells were obtained from the American Type Culture Collection (Manassas, Va.). Cells were cultured in RPMI 1640 medium (Gibco, Grand Island, N.Y.) supplemented with 10% heat-inactivated fetal bovine serum (FBS) (Gibco, Grand Island, N.Y.), 50 U/mL penicillin and 50 μg/mL streptomycin (Sigma, St. Louis, Mo.) at 37° C. in humidified 95% air and 5% CO₂.

Flow Cytometry Analysis of CD24 Positive MM Cells in Clinical Samples

Clinical bone marrow samples were obtained from MM patients in Huntsman Cancer Institute, University of Utah according to the ARUP protocol 25009 (n=11) and in the Nanjing Medical University, China (n=48). Studies were approved by the Institutional Review Board of the University of Utah and the Nanjing Medical University Informed consent was obtained in accordance with the Declaration of Helsinki.

Flow cytometric analysis was carried out on fresh bone marrow samples. All flow cytometric analyses were carried out on a Navios flow cytometer (Beckman Coulter, CA, USA). Bone marrow was stained with the following nine-color combination of mAsa, CD138/CD38/k/l/CD56/CD19/CD45/CD117/CD24 or three-color combination of mAbs, CD38/CD138/CD24 (BD biosciences, CA, USA), respectively. Data analysis was carried out with Kaluza software (Beckman Coulter, CA, USA), and MM cells were delineated using forward scatter/side scatter (FS/SS) and CD38/CD138 dot plots, after subgating on CD24 positive MM cells.

Western Blotting

Total or nuclear proteins were isolated with the Mammalian Cell Extraction Kit or Nuclear/Cytosol Fractionation Kit, respectively (BioVision, Mountain View, Calif.). Cell lysates were equally loaded onto 4-12% gels, electrophoresed, and transferred to nitrocellulose membranes. After blocking with 5% nonfat milk in Tris-buffered saline (TBS) containing 0.05% Tween-20, the membranes were incubated with the indicated primary antibodies overnight at 4° C. Protein bands were visualized using HRP-conjugated secondary antibodies and SuperSignal West Pico (Pierce). β-actin, GAPDH and histone H2b was used as an internal control.

Quantitative Real-Time PCR

Total RNA was extracted with an RNeasy RNA isolation kit (Qiagen). Complementary DNA was synthesized using Iscript reverse transcription kit according to the manufacturer's instructions (Bio-Rad). Quantitative Real-time PCR primers were purchased from Integrated DNA Technologies (Coralville, Iowa). Real-time quantitative PCRs were performed with SYBR Green Super Mixture Reagents (Bio-Rad) on the CFX connect real-time system (Bio-Rad). GAPDH transcript levels were used to normalize the amount of target cDNA.

Colony Formation Assay

Clonogenic growth was evaluated by seeding 2,000 cells or 10,000 primary cells in 0.5 mL MethoCult™ H4535 Enriched without EPO medium (Stem Cell Technologies, Vancouver, Canada) in a 12-well plate. The cells were incubated at 37° C. and 5% CO₂ and fed with RPM11640 medium containing StemSpan™ CC1100 Cytokine cocktail for expansion of human hematopoietic cells (Stem Cell Technologies) twice per week. All plates were taken pictures under inverted microscope and colonies consisting of more than 40 cells were scored. For serial re-plating, cells were eluted from the methylcellulose, washed, counted and replated in the MethoCult™ H4535 medium as described above.

Transcription Factor (TF) Activation Profiling Analysis

Each array assay was performed following the procedure described in the TF activation profiling plate array kit user manual (Signosis, Inc). 10 μg of nuclear extract was first incubated with the biotin labeled probe mix at room temperature for 30 min. The activated TFs were bound to the corresponding DNA binding probes. After the protein/DNA complexes were isolated from unbound probes, the bound probes were eluted and hybridized with the plate pre-coated with the capture oligonucleotides. The captured biotin-labeled probes were then detected with Streptavidin-HRP and subsequently measured with the chemiluminescent plate reader (Veritas microplate luminometer).

Xenograft Myeloma Mice

All animal work was performed in accordance with the guidelines of the Institutional Animal Care and local veterinary office and ethics committee of the University of Iowa under approved protocol. CD24⁺ or CD24⁺/STAT3 shRNA MM cells labeled with or without luciferase were injected subcutaneously into the each flank or by in vein of 6-8 weeks' NSG mice (Jackson laboratory, Bar Harbor, Me., USA) separately. Imaging was performed with a Xenogen IVIS 200 (Xenogen, Calif.). The mice were injected with 200 μl of 15 mg/mL D-leciferin intraperitoneally 15 min before imaging. The mice were sacrificed by CO₂ asphyxiation when subcutaneous tumors reached 20 mm in diameter.

Statistical Analysis

Two-tailed Student's t-test was used to evaluate two groups. One-way ANOVA was used to compare more than two groups. All values were expressed as mean±SD. Significance was defined as p<0.05.

### Example 1 References

1. Lapidot T, Sirard C, Vormoor J, et al. A cell initiating human acute myeloid leukaemia after transplantation into SCID mice. Nature 1994; 367:645-8.

2. Bonnet D, Dick J E. Human acute myeloid leukemia is organized as a hierarchy that originates from a primitive hematopoietic cell. Nat Med 1997; 3:730-7.

3. Al-Hajj M, Wicha M S, Benito-Hernandez A, Morrison S J, Clarke M F. Prospective identification of tumorigenic breast cancer cells. Proc Natl Acad Sci U S A 2003; 100:3983-8.

4. Ponti D, Costa A, Zaffaroni N, et al. Isolation and in vitro propagation of tumorigenic breast cancer cells with stem/progenitor cell properties. Cancer Res 2005; 65:5506-11.

5. Singh S K, Clarke I D, Terasaki M, et al. Identification of a cancer stem cell in human brain tumors. Cancer Res 2003; 63:5821-8.

6. Singh S K, Hawkins C, Clarke I D, et al. Identification of human brain tumour initiating cells. Nature 2004; 432:396-401.

7. Kondo T, Setoguchi T, Taga T. Persistence of a small subpopulation of cancer stem-like cells in the C6 glioma cell line. Proc Natl Acad Sci U S A 2004; 101:781-6.

8. Collins A T, Berry P A, Hyde C, Stower M J, Maitland N J. Prospective identification of tumorigenic prostate cancer stem cells. Cancer Res 2005; 65:10946-51.

9. Patrawala L, Calhoun T, Schneider-Broussard R, et al. Highly purified CD44+ prostate cancer cells from xenograft human tumors are enriched in tumorigenic and metastatic progenitor cells. Oncogene 2006; 25:1696-708.

10. Gibbs C P, Kukekov V G, Reith J D, et al. Stem-like cells in bone sarcomas: implications for tumorigenesis. Neoplasia 2005; 7:967-76.

11. Ricci-Vitiani L, Lombardi D G, Pilozzi E, et al. Identification and expansion of human colon-cancer-initiating cells. Nature 2007; 445:111-5.

12. Fang D, Nguyen T K, Leishear K, et al. A tumorigenic subpopulation with stem cell properties in melanomas. Cancer Res 2005; 65:9328-37.

13. Szotek P P, Pieretti-Vanmarcke R, Masiakos P T, et al. Ovarian cancer side population defines cells with stem cell-like characteristics and Mullerian Inhibiting Substance responsiveness. Proc Natl Acad Sci U S A 2006; 103:11154-9.

14. Hermann P C, Huber S L, Herrler T, et al. Distinct populations of cancer stem cells determine tumor growth and metastatic activity in human pancreatic cancer. Cell Stem Cell 2007; 1:313-23.

15. Olempska M, Eisenach P A, Ammerpohl O, Ungefroren H, Fandrich F, Kalthoff H. Detection of tumor stem cell markers in pancreatic carcinoma cell lines. Hepatobiliary Pancreat Dis Int 2007; 6:92-7.

16. Prince M E, Sivanandan R, Kaczorowski A, et al. Identification of a subpopulation of cells with cancer stem cell properties in head and neck squamous cell carcinoma. Proc Natl Acad Sci U S A 2007; 104:973-8.

17. Li X, Lewis M T, Huang J, et al. Intrinsic resistance of tumorigenic breast cancer cells to chemotherapy. J Natl Cancer Inst 2008; 100:672-9.

18. Bao S, Wu Q, McLendon R E, et al. Glioma stem cells promote radioresistance by preferential activation of the DNA damage response. Nature 2006; 444:756-60.

19. Rich J N. Cancer stem cells in radiation resistance. Cancer Res 2007; 67:8980-4.

20. Palumbo A, Anderson K. Multiple myeloma. N Engl J Med 2011; 364:1046-60.

21. Leung-Hagesteijn C, Erdmann N, Cheung G, et al. Xbps-negative tumor B cells and pre-plasmablasts mediate therapeutic proteasome inhibitor resistance in multiple myeloma. Cancer Cell 2013; 24:289-304.

22. Tanno T, Lim Y, Wang Q, et al. Growth differentiating factor 15 enhances the tumor-initiating and self-renewal potential of multiple myeloma cells. Blood 2014; 123:725-33.

23. Jakubikova J, Adamia S, Kost-Alimova M, et al. Lenalidomide targets clonogenic side population in multiple myeloma: pathophysiologic and clinical implications. Blood 2011; 117:4409-19.

24. Nara M, Teshima K, Watanabe A, et al. Bortezomib Reduces the Tumorigenicity of Multiple Myeloma via Downregulation of Upregulated Targets in Clonogenic Side Population Cells. PLoS One 2013; 8:e56954.

25. Hideshima T, Mitsiades C, Tonon G, Richardson P G, Anderson K C. Understanding multiple myeloma pathogenesis in the bone marrow to identify new therapeutic targets. Nat Rev Cancer 2007; 7:585-98.

26. Yaccoby S, Wezeman M J, Henderson A, et al. Cancer and the microenvironment: myeloma-osteoclast interactions as a model. Cancer Res 2004; 64:2016-23.

27. Bahlis N J, King A M, Kolonias D, et al. CD28-mediated regulation of multiple myeloma cell proliferation and survival. Blood 2007; 109:5002-10.

28. Rajkumar S V, Kyle R A. Multiple myeloma: diagnosis and treatment. Mayo Clin Proc 2005; 80:1371-82.

29. Matsui W, Wang Q, Barber J P, et al. Clonogenic multiple myeloma progenitors, stem cell properties, and drug resistance. Cancer Res 2008; 68:190-7.

30. Matsui W, Huff C A, Wang Q, et al. Characterization of clonogenic multiple myeloma cells. Blood 2004; 103:2332-6.

31. Boucher K, Parquet N, Widen R, et al. Stemness of B-cell progenitors in multiple myeloma bone marrow. Clin Cancer Res 2012; 18:6155-68.

32. Lim S C. CD24 and human carcinoma: tumor biological aspects. Biomed Pharmacother 2005; 59 Suppl 2:S351-4.

33. Jaggupilli A, Elkord E. Significance of CD44 and CD24 as cancer stem cell markers: an enduring ambiguity. Clin Dev Immunol 2012; 2012:708036.

34. Fang X, Zheng P, Tang J, Liu Y. CD24: from A to Z. Cell Mol Immunol 2010; 7:100-3.

35. Burgos-Ojeda D, Wu R, McLean K, et al. CD24+ Ovarian Cancer Cells Are Enriched for Cancer-Initiating Cells and Dependent on JAK2 Signaling for Growth and Metastasis. Mol Cancer Ther 2015; 14:1717-27.

36. Lee C J, Dosch J, Simeone D M. Pancreatic cancer stem cells. J Clin Oncol 2008; 26:2806-12.

37. Petkova N, Hennenlotter J, Sobiesiak M, et al. Surface CD24 distinguishes between low differentiated and transit-amplifying cells in the basal layer of human prostate. Prostate 2013; 73:1576-90.

38. Overdevest J B, Thomas S, Kristiansen G, Hansel D E, Smith S C, Theodorescu D. CD24 offers a therapeutic target for control of bladder cancer metastasis based on a requirement for lung colonization. Cancer Res 2011; 71:3802-11.

39. Karimi-Busheri F, Rasouli-Nia A, Zadorozhny V, Fakhrai H. CD24+/CD38− as new prognostic marker for non-small cell lung cancer. Multidiscip Respir Med 2013; 8:65.

40. Zhang C, Li C, He F, Cai Y, Yang H. Identification of CD44+CD24+ gastric cancer stem cells. J Cancer Res Clin Oncol 2011; 137:1679-86.

41. Badve S, Nakshatri H. Breast-cancer stem cells-beyond semantics. Lancet Oncol 2012; 13:e43-8.

42. Sagiv E, Starr A, Rozovski U, et al. Targeting CD24 for treatment of colorectal and pancreatic cancer by monoclonal antibodies or small interfering RNA. Cancer research 2008; 68:2803-12.

43. Reya T, Morrison S J, Clarke M F, Weissman I L. Stem cells, cancer, and cancer stem cells. Nature 2001; 414:105-11.

44. Zhan F, Barlogie B, Arzoumanian V, et al. Gene-expression signature of benign monoclonal gammopathy evident in multiple myeloma is linked to good prognosis. Blood 2007; 109:1692-700.

45. Kuranda K, Berthon C, Dupont C, et al. A subpopulation of malignant CD34+CD138+B7−H1+ plasma cells is present in multiple myeloma patients. Exp Hematol 2010; 38:124-31.

46. Yaccoby S, Barlogie B, Epstein J. Primary myeloma cells growing in SCID-hu mice: a model for studying the biology and treatment of myeloma and its manifestations. Blood 1998; 92:2908-13.

47. Yaccoby S, Epstein J. The proliferative potential of myeloma plasma cells manifest in the SCID-hu host. Blood 1999; 94:3576-82.

48. Kim D, Park C Y, Medeiros B C, Weissman I L. CD19−CD45 low/−CD38 high/CD138+ plasma cells enrich for human tumorigenic myeloma cells. Leukemia 2012; 26:2530-7.

49. Caligaris-Cappio F, Bergui L, Tesio L, et al. Identification of malignant plasma cell precursors in the bone marrow of multiple myeloma. J Clin Invest 1985; 76:1243-51.

50. Duperray C, Boiron J M, Boucheix C, et al. The CD24 antigen discriminates between pre-B and B cells in human bone marrow. J Immunol 1990; 145:3678-83.

51. Duperray C, Bataille R, Boiron J M, et al. No expansion of the pre-B and B-cell compartments in the bone marrow of patients with multiple myeloma. Cancer Res 1991; 51:3224-8.

52. Boyer L A, Lee T I, Cole M F, et al. Core transcriptional regulatory circuitry in human embryonic stem cells. Cell 2005; 122:947-56.

53. Orkin S H, Wang J, Kim J, et al. The transcriptional network controlling pluripotency in ES cells. Cold Spring Harb Symp Quant Biol 2008; 73:195-202.

54. Wang J, Rao S, Chu J, et al. A protein interaction network for pluripotency of embryonic stem cells. Nature 2006; 444:364-8.

55. Takahashi K, Tanabe K, Ohnuki M, et al. Induction of pluripotent stem cells from adult human fibroblasts by defined factors. Cell 2007; 131:861-72.

56. Hanna J, Saha K, Pando B, et al. Direct cell reprogramming is a stochastic process amenable to acceleration. Nature 2009; 462:595-601.

57. Franqui-Machin R, Wendlandt E B, Janz S, Zhan F, Tricot G. Cancer stem cells are the cause of drug resistance in multiple myeloma: fact or fiction? Oncotarget 2015; 6:40496-506.

Although the foregoing specification and examples fully disclose and enable the present invention, they are not intended to limit the scope of the invention, which is defined by the claims appended hereto.

All publications, patents and patent applications are incorporated herein by reference. While in the foregoing specification this invention has been described in relation to certain embodiments thereof, and many details have been set forth for purposes of illustration, it will be apparent to those skilled in the art that the invention is susceptible to additional embodiments and that certain of the details described herein may be varied considerably without departing from the basic principles of the invention.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating cancer in a patient comprising administering an effective amount of a therapeutic agent to the patient, wherein the cancer was determined to contain cells comprising cell marker CD24⁺.

2. A method of treating a cancer cell comprising cell marker CD24⁺, the method comprising administering to the cell a therapeutic agent.

3. The method of claim 1, wherein the cells further comprise cell marker CD38⁺ and/or are CD45 negative.

4. The method of claim 1, wherein the cancer is multiple myeloma.

5. The method of claim 1, wherein the therapeutic agent is a STAT3 inhibitor.

6. The method of claim 1, wherein the therapeutic agent is a ligand of CD24.

7. The method of claim 6, wherein the ligand of CD24 is a P-selectin antibody.

8. A method for predicting whether a cancer will respond to a therapeutic agent that targets a protein in the PBK pathway, the method comprising
(i) obtaining a test sample from a subject, wherein said test sample is from the cancer;
(ii) assaying the test sample for the presence of cell marker CD24⁺; and
(iii) using the presence of the cell marker CD24⁺ to indicate that the cancer will respond to the therapeutic agent.

9. The method of claim 8, wherein the cells further comprise cell marker CD38⁺ and/or are CD45 negative.

10. The method of claim 8, wherein the cancer is multiple myeloma.

11. The method of claim 8, wherein the assay is flow cytometry.

12. The method of claim 8, further comprising administering to a human subject having the cancer an effective amount of a therapeutic agent.

13. The method of claim 8, wherein the therapeutic agent is a STAT3 inhibitor.

14. The method of claim 8, wherein the therapeutic agent is a ligand of CD24.

15. The method of claim 14, wherein the ligand of CD24 is a P-selectin antibody.

16. The method of claim 2, wherein the cells further comprise cell marker CD38⁺ and/or are CD45 negative.

17. The method of claim 16, wherein the cancer is multiple myeloma.

18. The method of claim 16, wherein the therapeutic agent is a STAT3 inhibitor.

19. The method of claim 16, wherein the therapeutic agent is a ligand of CD24.

20. The method of claim 19, wherein the ligand of CD24 is a P-selectin antibody.
